SEQUENCE LISTING (52 sequences)

SEQ ID NO: 1 - DNA, 360 bp, Artificial Sequence
  Synthetic sequence Anti-STII mAb 3E8  VH
gaggtgcagctggtggagactgggggaggctttgtgaagcctggaggctccctgaaactctcctgtgcagcctctggattcactttcagtagttatggcatgtcttgggttcgccagactccggagaagaggctggagtgggtcgcagccatcaccagtgatggcggtggcacccactatccagatactgtgaagggccgattcaccatctccagagactttgccaaaaacaccctgtacctgcagatgagcagtctgaggtctgaggacacagcctggtatttctgtgcaagacatgagccccgactgatagcctggtttgctcactggggccaaggaactctggtcactgtctctgca

SEQ ID NO: 2 - protein, 120 aa, Artificial Sequence
  Synthetic sequence Anti-STII mAb 3E8  VH
EVQLVETGGGFVKPGGSLKLSCAASGFTFSSYGMSWVRQTPEKRLEWVAAITSDGGGTHYPDTVKGRFTISRDFAKNTLYLQMSSLRSEDTAWYFCARHEPRLIAWFAHWGQGTLVTVSA

SEQ ID NO: 3 - protein, 112 aa, Artificial Sequence
  Synthetic sequence Anti-STII mAb 3E8 VL
DVLMTQTPLSLPVSLGDQASISCRSSQSIVHSNGYTYLEWYLQKPGQSPKLLIYEVSNRFSGVPDRFSGSGSGTDFTLKIIRVEAEDLGVYYCFQGSHVPWTFGGGTKLEIK

SEQ ID NO: 4 - DNA, 337 bp, Artificial Sequence
  Synthetic sequence Anti-STII mAb 3E8 VL
gatgttttgatgacccaaactccactctccctgcctgtcagtcttggagatcaagcctctatctcttgcagatctagtcagagcattgttcatagtaatggatacacctatttagaatggtacctgcagaaaccaggccagtctccaaagctcctgatctacgaagtttccaaccgattttctggggtcccagacaggttcagtggcagtggatcagggacagatttcacactcaagatcatcagagtggaggctgaggatctgggagtttattattgctttcaaggttcacatgttccgtggacgttcggtggaggcaccaagctggaaatcaaac

SEQ ID NO: 5 - protein, 247 aa, Artificial Sequence
  Synthetic sequence Anti-STII 3E8 scFv
EVQLVETGGGFVKPGGSLKLSCAASGFTFSSYGMSWVRQTPEKRLEWVAAITSDGGGTHYPDTVKGRFTISRDFAKNTLYLQMSSLRSEDTAWYFCARHEPRLIAWFAHWGQGTLVTVSAGGGGSGGGGSGGGGSDVLMTQTPLSLPVSLGDQASISCRSSQSIVHSNGYTYLEWYLQKPGQSPKLLIYEVSNRFSGVPDRFSGSGSGTDFTLKIIRVEAEDLGVYYCFQGSHVPWTFGGGTKLEIK

SEQ ID NO: 6 - protein, 247 aa, Artificial Sequence
  Synthetic sequence  Anti-STII 3E8 scFv
DVLMTQTPLSLPVSLGDQASISCRSSQSIVHSNGYTYLEWYLQKPGQSPKLLIYEVSNRFSGVPDRFSGSGSGTDFTLKIIRVEAEDLGVYYCFQGSHVPWTFGGGTKLEIKGGGGSGGGGSGGGGSEVQLVETGGGFVKPGGSLKLSCAASGFTFSSYGMSWVRQTPEKRLEWVAAITSDGGGTHYPDTVKGRFTISRDFAKNTLYLQMSSLRSEDTAWYFCARHEPRLIAWFAHWGQGTLVTVSA

SEQ ID NO: 7 - DNA, 360 bp, Artificial Sequence
  Synthetic sequence  Anti-STII mAb 5G2 VH
caggttcaactgcagcagtctggagctgagctggcgaggccaggggcttcagtgaagctgtcctgcacggcttctggatacaccttcacaagctatggtataacctgggtgaggcagagaactggacagggccttgagtggattggagagatttttcctggaagtggtgatacttcctacggtgagaaattcaagggccaggccacactgactacagacaaatcctccagcacagcctacatgcagctcagcagcctgacatctgaggactctgcagtctatttctgtgcaagacgctataggtacatttaccatgctatggactactggggtcaaggaacctcagtcaccgtctcctca

SEQ ID NO: 8 - protein, 120 aa, Artificial Sequence
  Synthetic sequence Anti-STII mAb 5G2 VH
QVQLQQSGAELARPGASVKLSCTASGYTFTSYGITWVRQRTGQGLEWIGEIFPGSGDTSYGEKFKGQATLTTDKSSSTAYMQLSSLTSEDSAVYFCARRYRYIYHAMDYWGQGTSVTVSS

SEQ ID NO: 9 - DNA, 336 bp, Artificial Sequence
  Synthetic sequence Anti-STII mAb 5G2 VL
gatattttgatgacccaaactccactctccctgcctgtcagtcttggagatcaagcctccatctcttgcagatctagtcagagcattgtacatagtaatggcaacacctatttagaatggtacctgcagaaaccaggccagtctccaaagctcctgatctacaaagtttccaaccgattttctggggtcccagacaggttcagtggcagtggatcagggacagatttcacactcaagatccgcagagtggaggctgaggatctgggagtttattactgctttcaaggttcacatgttccgctcacgttcggtgctgggaccaagctggagctgaaa

SEQ ID NO: 10 - protein, 112 aa, Artificial Sequence
  Synthetic sequence Anti-STII mAb 5G2 VL
DILMTQTPLSLPVSLGDQASISCRSSQSIVHSNGNTYLEWYLQKPGQSPKLLIYKVSNRFSGVPDRFSGSGSGTDFTLKIRRVEAEDLGVYYCFQGSHVPLTFGAGTKLELK

SEQ ID NO: 11 - protein, 247 aa, Artificial Sequence
  Synthetic sequence Anti-STII 5G2 scFv
QVQLQQSGAELARPGASVKLSCTASGYTFTSYGITWVRQRTGQGLEWIGEIFPGSGDTSYGEKFKGQATLTTDKSSSTAYMQLSSLTSEDSAVYFCARRYRYIYHAMDYWGQGTSVTVSSGGGGSGGGGSGGGGSDILMTQTPLSLPVSLGDQASISCRSSQSIVHSNGNTYLEWYLQKPGQSPKLLIYKVSNRFSGVPDRFSGSGSGTDFTLKIRRVEAEDLGVYYCFQGSHVPLTFGAGTKLELK

SEQ ID NO: 12 - protein, 247 aa, Artificial Sequence
  Synthetic sequence Anti-STII 5G2 scFv
DILMTQTPLSLPVSLGDQASISCRSSQSIVHSNGNTYLEWYLQKPGQSPKLLIYKVSNRFSGVPDRFSGSGSGTDFTLKIRRVEAEDLGVYYCFQGSHVPLTFGAGTKLELKGGGGSGGGGSGGGGSQVQLQQSGAELARPGASVKLSCTASGYTFTSYGITWVRQRTGQGLEWIGEIFPGSGDTSYGEKFKGQATLTTDKSSSTAYMQLSSLTSEDSAVYFCARRYRYIYHAMDYWGQGTSVTVSS

SEQ ID NO: 13 - DNA, 360 bp, Artificial Sequence
  Synthetic sequence Anti-STII mAb 4E2 VH
caggttcaactgcagcagtctggagctgagctggcgaggccaggggcttcagtgaagctgtcctgcacggcttctggatacaccttcacaagctatggtataacctgggtgaggcagagaactggacagggccttgagtggattggagagatttttcctggaagtggtgatacttcctacggtgagaaattaaagggccaggccacactgactacagacaaatcctccagcacagcctacatgcagctcagcagcctgacatctgaggactctgcagtctatttctgtgcaagacgctataggtacatttaccatgctatggactactggggtcaaggaacctcagtcaccgtctcctca

SEQ ID NO: 14 - protein, 120 aa, Artificial Sequence
  Synthetic sequence Anti-STII mAb 4E2 VH
QVQLQQSGAELARPGASVKLSCTASGYTFTSYGITWVRQRTGQGLEWIGEIFPGSGDTSYGEKLKGQATLTTDKSSSTAYMQLSSLTSEDSAVYFCARRYRYIYHAMDYWGQGTSVTVSS

SEQ ID NO: 15 - DNA, 336 bp, Artificial Sequence
  Synthetic sequence Anti-STII mAb 4E2  VL
gatattttgatgacccaaactccactctccctgcctgtcagtcttggagatcaagcctccatctcttgcagatctagtcagagcattgtacatagtaatggcaacacctatttagagtggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaa

SEQ ID NO: 16 - protein, 112 aa, Artificial Sequence
  Synthetic sequence Anti-STII mAb 4E2  VL
DILMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 17 - protein, 247 aa, Artificial Sequence
  Synthetic sequence Anti-STII 4E2 scFv
QVQLQQSGAELARPGASVKLSCTASGYTFTSYGITWVRQRTGQGLEWIGEIFPGSGDTSYGEKLKGQATLTTDKSSSTAYMQLSSLTSEDSAVYFCARRYRYIYHAMDYWGQGTSVTVSSGGGGSGGGGSGGGGSDILMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 18 - protein, 247 aa, Artificial Sequence
  Synthetic sequence Anti-STII 4E2 scFv
DILMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELKGGGGSGGGGSGGGGSQVQLQQSGAELARPGASVKLSCTASGYTFTSYGITWVRQRTGQGLEWIGEIFPGSGDTSYGEKLKGQATLTTDKSSSTAYMQLSSLTSEDSAVYFCARRYRYIYHAMDYWGQGTSVTVSS

SEQ ID NO: 19 - protein, 8 aa, Artificial Sequence
  Synthetic sequence Strep-Tag II
WSHPQFEK

SEQ ID NO: 20 - protein, 10 aa, Artificial Sequence
  Synthetic sequence Gly4Ser)2 linker
GGGGSGGGGS

SEQ ID NO: 21 - protein, 8 aa, Artificial Sequence
  Synthetic sequence Gly3Ser)2 linker
GGGSGGGS

SEQ ID NO: 22 - protein, 13 aa, Artificial Sequence
  Synthetic sequence 3E8 HCDR1
AASGFTFSSYGMS

SEQ ID NO: 23 - protein, 10 aa, Artificial Sequence
  Synthetic sequence 3E3 HCDR2
AITSDGGGTH

SEQ ID NO: 24 - protein, 13 aa, Artificial Sequence
  Synthetic sequence 3E8 HCDR3
ARHEPRLIAWFAH

SEQ ID NO: 25 - protein, 16 aa, Artificial Sequence
  Synthetic sequence 3E8 LCDR1
RSSQSIVHSNGYTYLE

SEQ ID NO: 26 - protein, 8 aa, Artificial Sequence
  Synthetic sequence 3E8 LCDR2
YEVSNRFS

SEQ ID NO: 27 - protein, 9 aa, Artificial Sequence
  Synthetic sequence 3E8 LCDR3
FQGSHVPWT

SEQ ID NO: 28 - protein, 13 aa, Artificial Sequence
  Synthetic sequence 5G2 HCDR1
TASGYTFTSYGIT

SEQ ID NO: 29 - protein, 10 aa, Artificial Sequence
  Synthetic sequence 5G2 HCDR2
EIFPGSGDTS

SEQ ID NO: 30 - protein, 13 aa, Artificial Sequence
  Synthetic sequence 5G2 HCDR3
ARRYRYIYHAMDY

SEQ ID NO: 31 - protein, 16 aa, Artificial Sequence
  Synthetic sequence 5G2 LCDR1
RSSQSIVHSNGNTYLE

SEQ ID NO: 32 - protein, 8 aa, Artificial Sequence
  Synthetic sequence 5G2 LCDR2
YKVSNRFS

SEQ ID NO: 33 - protein, 9 aa, Artificial Sequence
  Synthetic sequence 5G2 LCDR3
FQGSHVPLT

SEQ ID NO: 34 - protein, 13 aa, Artificial Sequence
  Synthetic sequence 4E2 HCDR1
TASGYTFTSYGIT

SEQ ID NO: 35 - protein, 10 aa, Artificial Sequence
  Synthetic sequence 4E2 HCDR2
EIFPGSGDTS

SEQ ID NO: 36 - protein, 13 aa, Artificial Sequence
  Synthetic sequence 4E2  HCDR3
ARRYRYIYHAMDY

SEQ ID NO: 37 - protein, 16 aa, Artificial Sequence
  Synthetic sequence 4E2  LCDR1
RSSQSIVHSNGNTYLE

SEQ ID NO: 38 - protein, 8 aa, Artificial Sequence
  Synthetic sequence 4E2 LCDR2
YKVSNRFS

SEQ ID NO: 39 - protein, 9 aa, Artificial Sequence
  Synthetic sequence 4E2  LCDR3
FQGSHVPLT

SEQ ID NO: 40 - protein, 19 aa, Artificial Sequence
  Synthetic sequence P2A
ATNFSLLKQAGDVEENPGP

SEQ ID NO: 41 - protein, 22 aa, Artificial Sequence
  Synthetic sequence P2A mod
GSGATNFSLLKQAGDVEENPGP

SEQ ID NO: 42 - protein, 18 aa, Artificial Sequence
  Synthetic sequence T2A
EGRGSLLTCGDVEENPGP

SEQ ID NO: 43 - protein, 21 aa, Artificial Sequence
  Synthetic sequence T2A mod
GSGEGRGSLLTCGDVEENPGP

SEQ ID NO: 44 - protein, 21 aa, Artificial Sequence
  Synthetic sequence E2A
QCTNYALLKLAGSDVESNPGP

SEQ ID NO: 45 - protein, 24 aa, Artificial Sequence
  Synthetic sequence E2A mod
GSGQCTNYALLKLAGSDVESNPGP

SEQ ID NO: 46 - protein, 22 aa, Artificial Sequence
  Synthetic sequence F2A
VKQTLNFDLLKLAGDVESNPGP

SEQ ID NO: 47 - protein, 25 aa, Artificial Sequence
  Synthetic sequence F2A mod
GSGVKQTLNFDLLKLAGDVESNPGP

SEQ ID NO: 48 - protein, 8 aa, Artificial Sequence
  Synthetic sequence Strep-tag
WRHPQFGG

SEQ ID NO: 49 - protein, 11 aa, Artificial Sequence
  Synthetic sequence Gly3Ser)2Gly2Ser linker
GGGSGGGSGGS

SEQ ID NO: 50 - protein, 10 aa, Artificial Sequence
  Synthetic sequence combination ST peptide
  ACETYLATION 1
  MOD_RES 2: dPEG4
  AMIDATION 10
CNWSHPQFEK

SEQ ID NO: 51 - protein, 11 aa, Artificial Sequence
  Synthetic sequence combination ST peptide
  AMIDATION 11
CGNWSHPQFEK

SEQ ID NO: 52 - protein, 13 aa, Artificial Sequence
  Synthetic sequence combination ST peptide
CGNWSHPQFEKGC

(19) United States
(12) Patent Application Publication
(10) Pub. No.: US 2020/0262894 A1
(43) Pub. Date: Aug. 20, 2020

(54) STREP-TAG SPECIFIC BINDING PROTEINS AND USES THEREOF

(71) Applicant: FRED HUTCHINSON CANCER RESEARCH CENTER, Seattle, WA (US)

(72) Inventors: Lingfeng LIU, Seattle, WA (US); Stanley R. RIDDELL, Sammamish, WA (US); Benjamin HOFFSTROM, Seattle, WA (US)

(21) Appl. No.: 16/645,428

(22) PCT Filed: Sep. 6, 2018
(86) PCT No.: PCT/US2018/049808

§ 371 Date: Mar. 6, 2020

Related U.S. Application Data
(60) Provisional application No. 62/555,017, filed on Sep. 6, 2017.

(51) Int. Cl. C07K 16/12; C12N 5/0783; G01N 33/569; A61K 49/00; A61K 39/00

(52) U.S. Cl. CPC C07K 16/1292; C12N 5/0638; G01N 33/56972; A61K 49/00; A61K 2039/5158; C07K 2317/565; C07K 2319/03; A61K 2039/505; A61K 2039/5156; A61K 39/001112

(57) ABSTRACT

The present disclosure provides immunoglobulin binding proteins and fusion proteins that specifically bind to a strep tag peptide, such as a peptide having the amino acid sequence set forth in SEQ ID NO: 19. Also provided are methods for using the disclosed compositions in a cellular immunotherapy wherein the therapeutic cells express a tag peptide.

### CROSS-REFERENCE TO RELATED APPLICATION(S)

This application claims the priority benefit of U.S. Patent Application No. 62/555,017, filed Sep. 6, 2017, which is incorporated herein by reference for all purposes as if fully set forth herein.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_451WO_SEQUENCE_LISTING.txt. The text file is 28.9 KB, was created on Sep. 3, 2018, and is being submitted electronically via EFS-Web.

### BACKGROUND

Recombinant proteins and cells expressing the same are commonly detected, sorted, and purified using synthetic tag peptides that are fused to the recombinant proteins. For example, the synthetic Strep®-Tag II peptide can be readily fused to a protein of interest and binds to the engineered streptavidin derivative Strep-Tactin® with high affinity. The Strep-Tag® system allows isolation and affinity purification of Strep-tag-labeled proteins and cells via binding to a Strep-Tactin®-containing substrate, which is typically a magnetic nanobead or a resin.

However, Strep-Tag®-binding reagents with additional functionalities are needed in order to more fully exploit the potential of tagging target molecules for in vitro and in vivo applications, such as detecting and manipulating tagged proteins and cells used in immunotherapies. Presently disclosed embodiments address these needs and provide other related advantages.

### BRIEF DESCRIPTION OF THE DRAWINGS

FIGS. 1A and 1B show characterization of murine anti-Strep®-Tag II (STII) monoclonal antibodies that bind STII-tagged CAR T cells. (A) Flow cytometry data showing specific binding by anti-STII monoclonal antibodies to STII-tagged CAR T cells. (B) IsoStrip™ indicating isotypes of 5G2 mAb and 4E2 mAb.

FIGS. 2A-2D show data from in vivo experiments where B cell-depleted mice receiving STII-tagged anti-CD19 CAR T cells were administered anti-STII monoclonal antibodies of the present disclosure. (A) Experimental scheme for B cell rescue using anti-STII mAb. (B) Flow cytometry data showing expression of STII-tagged CARs by mouse T cells 7 days before infusion. Top row: expression of CAR constructs containing one (1) STII tag. Middle row: CARs containing three (3) STII tags. Bottom row: non-transduced cells. Cells were stained for CD19, CD45.1, EGFR, and STII. (C) Flow cytometry data showing B cell recovery in mice treated with anti-CD19-1STII CAR T cells followed by anti-STII mAbs. (D) Flow cytometry data showing B cell recovery in mice treated with anti-CD19-3STII CAR T cells followed by anti-STII mAbs.

FIG. 3A provides exemplary flow cytometry data showing expansion of tagged CAR T cells using (from top to bottom) control microbeads; microbeads coated with 0.1, 0.3, or 0.5 μg anti-STII mAb; or microbeads coated with anti-STII mAb/anti-CD28mAb (both 0.3 μg). FIG. 3B shows IL-2 (top) and IFN-γ (bottom) release by cells contacted with microbeads coated with the indicated amount of antibody.

FIG. 4A provides data showing expansion of STII-tagged CAR T cells following 1, 2, or 3 (left to right) rounds of stimulation with coated microbeads as indicated in the key. FIG. 4B shows expression of the indicated T cell markers by tagged CD8⁺ and CD4⁺ CAR T cells pre-stimulation and following 1, 2, or 3 rounds of stimulation. Cells were stained using antibodies for: STII; CD45RO; CD62L; CD28L; CTLA4; and PD1.

### DETAILED DESCRIPTION

The instant disclosure provides compositions and methods for identifying, sorting, tracking, and selectively modulating recombinant proteins and host cells that comprise or express Strep-Tag II (WSHPQFEK, SEQ ID NO:19). In certain aspects, immunoglobulin binding proteins, fusion proteins, and host cells expressing the same are provided that are useful in modulating tagged immune cells for, for example, adoptive cell therapies.

By way of background, adoptive transfer of genetically modified T cells has emerged as a potent therapy for various malignancies. The most widely employed strategy has been infusion of patient-derived T cells expressing chimeric antigen receptors (CARs) targeting tumor associated antigens. This approach can be used to target T cells to a cell surface antigen, circumvent loss of major histocompatibility complex as a tumor escape mechanism, and employ a single vector construct to treat any patient, regardless of human leukocyte antigen (HLA) haplotype. For example, CAR clinical trials for B-cell non-Hodgkin's lymphoma (NHL) have, to date, targeted CD19, CD20, or CD22 antigens that are expressed on malignant lymphoid cells as well as on normal B cells (Brentjens et al., Sci Transl Med 2013; 5(177):177ra38; Haso et al., Blood 2013; 121(7):1165-74; James et al., J Immunol 2008; 180(10):7028-38; Kalos et al., Sci Transl Med 2011; 3 (95): 95ra73; Kochenderfer et al., J Clin Oncol 2015; 33(6):540-9; Lee et al., Lancet 2015; 385(9967):517-28; Porter et al., Sci Transl 25 Med 2015; 7(303):303ra139; Savoldo et al., J Clin Invest 2011; 121(5):1822-6; Till et al., Blood 2008; 112(6):2261-71; Till et al., Blood 2012; 119(17):3940-50; Coiffier et al., N Engl J Med 2002; 346(4):235-42).

Tools for adoptive cell therapies include tagged chimeric effector molecules, such as those described in PCT Publication No. WO 2015/095895 (the tagged effector molecules of which are herein incorporated by reference). In this disclosure, immunoglobulin binding proteins were produced that were shown to be capable of identifying and modulating (e.g., activating, inducing to proliferate, impairing, or killing) cells expressing such tagged molecules with high specificity and fidelity.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers. A polypeptide may further contain other components (e.g., covalently bound), such as a tag, a label, a bioactive molecule, or any combination thereof. In certain embodiments, a polypeptide may be a fragment. As used herein, a “fragment” means a polypeptide that is lacking one or more amino acids that are found in a reference sequence. A fragment can comprise a binding domain, antigen, or epitope found in a reference sequence. A fragment of a reference polypeptide can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of amino acids of the amino acid sequence of the reference sequence.

As used herein, “fusion protein” refers to a protein that, in a single chain, has at least two distinct domains, wherein the domains are not naturally found together in a protein. A polynucleotide encoding a fusion protein may be constructed using PCR, recombinantly engineered, or the like, or such fusion proteins can be synthesized. A fusion protein may further contain other components, such as a tag, a linker, or a transduction marker. In certain embodiments, a fusion protein expressed or produced by a host cell (e.g., a T cell) locates to a cell surface, where the fusion protein is anchored to the cell membrane (e.g., via a transmembrane domain) and comprises an extracellular portion (e.g., containing a binding domain) and an intracellular portion (e.g., containing a signaling domain, effector domain, co-stimulatory domain or combinations thereof).

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single- or double-stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridizes to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a fusion protein or a binding domain thereof having a functionality described herein, such as specifically binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the encoded parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{d}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release).

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As used herein, the term “host” refers to a cell (e.g., T cell, Chinese Hamster Ovary (CHO) cell, HEK293 cell, B cell, or the like) or microorganism targeted for genetic modification with a heterologous nucleic acid molecule to produce a polypeptide of interest (e.g., a fusion protein of the present disclosure). In certain embodiments, a host cell may optionally already possess or be modified to include other genetic modifications that confer desired properties related or unrelated to, e.g., biosynthesis of the heterologous protein (e.g., inclusion of a detectable marker; deleted, altered or truncated endogenous TCR; or increased co-stimulatory factor expression).

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g, Geurts et al., Mol. Ther. 8:108, 2003: Mates et al., Nat. Genet. 41:753 (2009)). Exemplary vectors are those capable of autonomous replication (episomal vector) or expression of nucleic acid molecules to which they are linked (expression vectors).

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. Thus, depending upon the source of an original population of cells subjected to an enriching process, a mixture or composition may contain 30%, 35%, 40%, 45%, 50%, 55%, 60%, 65%, 70%, 75%, 80%, 85%, 90%, 91%, 92%, 93%, 94%, 95%, 96%, 97%, 98%, or 99% or more (in number or count) of the “enriched” cells. Cells subjected to a depleting process can result in a mixture or composition containing 50%, 45%, 40%, 35%, 30%, 25%, 20%, 15%, 10%, 9%, 8%, 7%, 6%, 5%, 4%, 3%, 2%, or 1% percent or less (in number or count) of the “depleted” cells. In certain embodiments, amounts of a certain cell type in a mixture will be enriched and amounts of a different cell type will be depleted, such as enriching for CD4⁺ cells while depleting CD8⁺ cells, or enriching for CD62L⁺ cells while depleting CD62L⁻ cells, or combinations thereof.

“Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a host cell expressing a fusion protein of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

A “therapeutically effective amount” or “effective amount” of a fusion protein, or host cell expressing a fusion protein of this disclosure refers to an amount of fusion proteins or host cells sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient, such as two different fusion proteins (e.g., CARs) that specifically bind a tag peptide comprising the amino acid sequence shown in SEQ ID NO:19, or a fusion protein of the present.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event.

As used herein, “statistically significant” refers to a p value of 0.050 or less when calculated using the Students t-test and indicates that it is unlikely that a particular event or result being measured has arisen by chance.

As used herein, the term “adoptive immune therapy” or “adoptive immunotherapy” refers to administration of naturally occurring or genetically engineered, disease antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

### Immunoglobulin Binding Proteins

In certain aspects, the present disclosure provides an immunoglobulin binding protein comprising a binding domain that specifically binds to a strep-tag peptide. As used herein, the term “strep-tag peptide” (also referred to herein as a “strep-tag,” a “strep tag,” a “ST,” and a “tag peptide” (when the context clearly indicates as such and does not indicate a different type of peptide that is used to tag a protein of interest (e.g., Myc, His, or Flag)) means a peptide that is capable of specifically binding to streptavidin (which is a tetrameric protein purified from Streptomyces avidinii and is widely used in molecule biology protocols due to its high affinity for biotin) or to Streptactin®, which is an engineered mutein of streptavidin. Exemplary strep-tag peptides of the instant disclosure compete with biotin for binding to streptavidin or a mutein or variant thereof (e.g., Streptactin®) and include, for example, Strep® tag (WRHPQFGG, SEQ ID NO:48); Strep® Tag II (also referred to as “STII” herein, which consists of the amino acid sequence WSHPQFEK (SEQ ID NO:19)); and variants thereof, including those disclosed in, for example, Schmidt and Skerra, Nature Protocols, 2:1528-1535 (2007), U.S. Pat. No. 7,981,632; and PCT Publication No. WO 2015/067768, the strep-tag peptides, step-tag-peptide-containing polypeptides, and sequences of the same, are incorporated herein by reference.

In certain embodiments, an immunoglobulin binding protein comprises a binding domain that is capable of specifically binding to a strep-tag peptide, wherein the binding domain comprises a V_{H} domain and a V_{L} domain comprising CDRs, or variants thereof, according to monoclonal antibody 3E8, 5G2, or 4E2. In certain embodiments, the binding domain comprises: (a) a V_{L} domain comprising: (i) a CDR1 amino acid sequence shown in SEQ ID NO:25, or a variant thereof, a CDR2 amino acid sequence shown in SEQ ID NO:26, or a variant thereof, and a CDR3 amino acid sequence shown in SEQ ID NO:27, or a variant thereof; (ii) a CDR1 amino acid sequence shown in SEQ ID NO:31, or a variant thereof, a CDR2 amino acid sequence shown in SEQ ID NO:32, or a variant thereof, and a CDR3 amino acid sequence shown in SEQ ID NO:33, or a variant thereof; or (iii) a CDR1 amino acid sequence shown in SEQ ID NO:37, or a variant thereof, a CDR2 amino acid sequence shown in SEQ ID NO:38, or a variant thereof, and a CDR3 amino acid sequence shown in SEQ ID NO:39, or a variant thereof, and a V_{H} domain (which may, in embodiments, have at least about 80, 85, 90, 91, 92, 93, 94, 95, 96, 97, 98, 99, or 99.9% identity to the amino acid sequence shown in any one of SEQ ID NOs: 2, 8, or 14); or (b) a V_{H} domain comprising: (i) the CDR1 amino acid sequence shown in SEQ ID NO:22, or a variant thereof, the CDR2 amino acid sequence shown in SEQ ID NO:23, or a variant thereof, and the CDR3 amino acid sequence shown in SEQ ID NO:24, or a variant thereof; (ii) a CDR1 amino acid sequence shown in SEQ ID NO:28, or a variant thereof, a CDR2 amino acid sequence shown in SEQ ID NO:29, or a variant thereof, and the CDR3 amino acid sequence shown in SEQ ID NO:30, or a variant thereof; or (iii) the CDR1 amino acid sequence shown in SEQ ID NO:34, or a variant thereof, the CDR2 amino acid sequence shown in SEQ ID NO:35, or a variant thereof, and the CDR3 amino acid sequence shown in SEQ ID NO:36, or a variant thereof, and a V_{L} domain (which may, in embodiments, have at least about 80, 85, 90, 91, 92, 93, 94, 95, 96, 97, 98, 99, or 99.9% identity to the amino acid sequence shown in any one of SEQ ID NOs:3, 10, or 16); or (c) the V_{L} domain of (a) and the V_{H} domain of (b). In particular embodiments, the V_{H} domain comprises (i) the CDR1 amino acid sequence shown in SEQ ID NO: 22, (ii) the CDR2 amino acid sequence shown in SEQ ID NO:23, and (iii) the CDR3 amino acid sequence shown in SEQ ID NO:24; and the V_{L} domain comprises (iv) the CDR1 amino acid sequence shown in SEQ ID NO: 25, (v) the CDR2 amino acid sequence shown in SEQ ID NO:26, and (vi) the CDR3 amino acid sequence shown in SEQ ID NO:27.

In other embodiments, the V_{H} domain comprises (i) the CDR1 amino acid sequence shown in SEQ ID NO: 28, (ii) the CDR2 amino acid sequence shown in SEQ ID NO:29, and (iii) the CDR3 amino acid sequence shown in SEQ ID NO:30; and the V_{L} domain comprises (iv) the CDR1 amino acid sequence shown in SEQ ID NO: 31, (v) the CDR2 amino acid sequence shown in SEQ ID NO:32, and (vi) the CDR3 amino acid sequence shown in SEQ ID NO:33.

In other embodiments, the V_{H} domain comprises (i) the CDR1 amino acid sequence shown in SEQ ID NO: 34, (ii) the CDR2 amino acid sequence shown in SEQ ID NO:35, and (iii) the CDR3 amino acid sequence shown in SEQ ID NO:36; and the V_{L} domain comprises (iv) the CDR1 amino acid sequence shown in SEQ ID NO:37, (v) the CDR2 amino acid sequence shown in SEQ ID NO:38, and (vi) the CDR3 amino acid sequence shown in SEQ ID NO:39.

In any of the aforementioned embodiments or other embodiments disclosed herein, the strep-tag peptide comprises or consists of the amino acid sequence of SEQ ID NO:19.

A “binding domain” or “binding region,” as used herein, refers to a protein, polypeptide, oligopeptide, or peptide (e.g., antibody, receptor) or portion or fragment thereof that possesses the ability to specifically recognize and non-covalently associate with a target (e.g., antigen, ligand). A binding domain includes any naturally occurring, synthetic, semi-synthetic, or recombinantly produced binding partner for a biological molecule or another target of interest. Exemplary binding domains include immunoglobulin light and heavy chain variable regions (e.g., domain antibodies, sFv, single chain Fv fragment (scFv), Fab, F(ab′)₂), receptor ectodomains, or ligands. Immunoglobulin variable domains (e.g., scFv, Fab) are referred to herein as “immunoglobulin binding domains.” A variety of assays are known for identifying binding domains of the present disclosure that specifically bind a particular target, including Western blot, ELISA, and Biacore® analysis. In certain embodiments, the binding domain is chimeric, human, or humanized.

In certain embodiments, a binding domain is part of a larger polypeptide or protein and is referred to as a “binding protein.” An “immunoglobulin binding protein” or “immunoglobulin-like binding protein” refers to a polypeptide containing one or more immunoglobulin binding domains, wherein the polypeptide may be in the form of any of a variety of immunoglobulin-related protein scaffolds or structures, such as an antibody or an antigen binding fragment thereof, a scFv-Fc fusion protein, or a fusion protein comprising two or more of such immunoglobulin binding domains or other binding domains.

Sources of binding domains include antibody variable regions from various species, including human, rodent, avian, leporine, and ovine. Additional sources of binding domains include variable regions of antibodies from other species, such as camelid (from camels, dromedaries, or llamas; Ghahroudi et al., FEBS Letters 414: 521, 1997; Vincke et al., J. Biol. Chem. 284: 3273, 2009; Hamers-Casterman et al., Nature 363: 446, 1993 and Nguyen et al., J. Mol. Biol. 275: 413, 1998), nurse sharks (Roux et al., Proc. Nat'l. Acad. Sci. (USA) 95: 11804, 1998), spotted ratfish (Nguyen et al., Immunogenetics 54: 39, 2002), or lamprey (Herrin et al., Proc. Nat'l. Acad. Sci. (USA) 105: 2040,2008 and Alder et al., Nature Immunol. 9: 319, 2008). These antibodies can apparently form antigen-binding regions using only heavy chain variable region, i.e., these functional antibodies are homodimers of heavy chains only (referred to as “heavy chain antibodies”) (Jespers et al., Nature Biotechnol. 22: 1161, 2004; Cortez-Retamozo et al., Cancer Res. 64: 2853, 2004; Baral et al., Nature Med. 12: 580, 2006; and Barthelemy et al., J. Biol. Chem. 283: 3639, 2008).

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds (though it will be understood that heavy chain antibodies, which lack light chains, are still encompassed by the term “antibody”), as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen-binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The terms “V_{L}” and “V_{H}” refer to the variable binding region from an antibody light and heavy chain, respectively. The variable binding regions are made up of discrete, well-defined sub-regions known as “complementarity determining regions” (CDRs) and “framework regions” (FRs). The terms “complementarity determining region,” and “CDR,” are synonymous with “hypervariable region” or “HVR,” and are known in the art to refer to non-contiguous sequences of amino acids within TCR or antibody variable regions, which confer antigen specificity and/or binding affinity. In general, there are three CDRs in each variable region of an immunoglobulin binding protein; e.g., for antibodies, the V_{H} and V_{L} regions comprise six CDRs HCDR1, HCDR2, HCDR3; LCDR1, LCDR2, LCDR3). As used herein, a “variant” of a CDR refers to a functional variant of a CDR sequence having up to 1-3 amino acid substitutions, deletions, or combinations thereof. Immunoglobulin sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300).

“Antigen” or “Ag” as used herein refers to an immunogenic molecule that provokes an immune response. This immune response may involve antibody production, activation of the complement pathway, activation of specific immunologically competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” or “antigenic epitope” includes any molecule, structure, amino acid sequence, or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

As used herein, “specifically binds” or “specific for” refers to an association or union of a binding protein or a binding domain (or fusion protein thereof) to a target molecule (e.g., a tag peptide comprising or consisting of the amino acid sequence of WSHPQFEK, SEQ ID NO: 19) with an affinity or Kₐ (i.e., an equilibrium association constant of a particular binding interaction with units of 1/M) equal to or greater than 10⁵M⁻¹ (which equals the ratio of the on-rate [Kₒₙ] to the off rate [K_{off}] for this association reaction), while not significantly associating or uniting with any other molecules or components in a sample. Binding proteins or binding domains (or fusion proteins thereof) may be classified as “high-affinity” binding proteins or binding domains (or fusion proteins thereof) or as “low-affinity” binding proteins or binding domains (or fusion proteins thereof). “High-affinity” binding proteins or binding domains refer to those binding proteins or binding domains having a Kₐ of at least 10⁷ M⁻¹, at least 10⁸M⁻¹, at least 10⁹M⁻¹, at least 10¹⁰ M⁻¹, at least 10¹¹ M⁻¹, at least 10¹²M⁻¹, or at least 10¹³M⁻¹. “Low-affinity” binding proteins or binding domains refer to those binding proteins or binding domains having a Kₐ of up to 10⁷M⁻¹, up to 10⁶M⁻¹, or up to 10⁵M⁻¹. Alternatively, affinity may be defined as an equilibrium dissociation constant (K_{d}) of a particular binding interaction with units of M (e.g., 10⁻⁵ M to 10⁻¹³ M).

A variety of assays are known for identifying immunoglobulin binding proteins and binding domains of the present disclosure that specifically bind a particular target, as well as determining binding domain or binding protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known. In certain examples, apparent affinity for an immunoglobulin binding protein is measured by assessing binding to various concentrations of tetramers, for example, by flow cytometry using labeled tetramers. In some examples, apparent K_{d} of an immunoglobulin binding protein is measured using 2-fold dilutions of labeled tetramers at a range of concentrations, followed by determination of binding curves by non-linear regression, apparent K_{d} being determined as the concentration of ligand that yielded half-maximal binding.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype, into CH1, CH2, and CH3 (IgA, IgD, IgG), or CH1, CH2, CH3, and CH4 domains (IgE, IgM). A “Fab” (fragment antigen binding) is the part of an antibody that binds to antigen and includes the variable region and CH1 of the heavy chain linked to the light chain via an inter-chain disulfide bond.

In certain embodiments, the V_{L} domain of an immunoglobulin binding protein comprises an amino acid sequence that is at least 80, 85, 90, 91, 92, 93, 94, 95, 96, 97, 98, 99, or 99.9% identical to the amino acid sequence shown in any one of SEQ ID NOS:3, 10, and 16, and the V_{H} domain comprises an amino acid sequence that is at least 80, 85, 90, 91, 92, 93, 94, 95, 96, 97, 98, 99, or 99.9% identical to the amino acid sequence shown in any one of SEQ ID NOS: 2, 8, and 14. In further embodiments, the V_{L} of the immunoglobulin binding protein comprises or consists of the amino acid sequence shown in any one of SEQ ID NOS:3, 10, and 16, and the V_{H} comprises or consists of the amino acid sequence shown in any one of SEQ ID NOS:2, 8, and 14. In particular embodiments, an immunoglobulin binding protein comprises:

(i) a V_{L} domain comprising or consisting of the amino acid sequence shown in SEQ ID NO:2, and a V_{H} domain comprising or consisting of the amino acid sequence shown in SEQ ID NO:3; (ii) a V_{L} domain comprising or consisting of the amino acid sequence shown in SEQ ID NO:10, and a V_{H} domain comprising or consisting of the amino acid sequence shown in SEQ ID NO:8; or a V_{L} domain comprising or consisting of the amino acid sequence shown in SEQ ID NO:16, and a V_{H} domain comprising or consisting of the amino acid sequence shown in SEQ ID NO:14.

As used herein, “Fc region portion” refers to the heavy chain constant region segment of the Fc fragment (the “fragment crystallizable” region or Fc region) from an antibody, which can in include one or more constant domains, such as CH2, CH3, CH4, or any combination thereof. In certain embodiments, an Fc region portion includes the CH2 and CH3 domains of an IgG, IgA, or IgD antibody or any combination thereof, or the CH3 and CH4 domains of an IgM or IgE antibody, and any combination thereof. In other embodiments, a CH2CH3 or a CH3CH4 structure has sub-region domains from the same antibody isotype and are human, such as human IgG1, IgG2, IgG3, IgG4, IgA1, IgA2, IgD, IgE, or IgM (e.g., CH2CH3 from human IgG1). By way of background, an Fc region is responsible for the effector functions of an immunoglobulin, such as ADCC (antibody-dependent cell-mediated cytotoxicity), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al., Nature 337: 525, 1989). In certain embodiments, an Fc region portion found in immunoglobulin-like binding proteins of the present disclosure will be capable of mediating one or more of these effector functions, or will lack one or more or all of these activities by way of, for example, one or more mutations known in the art. For example, amino acid modifications (e.g., substitutions) to modify (e.g., improve, reduce, or ablate) Fc functionalities include the T250Q/M428L; M252Y/S254T/T256E; H433K/N434F; M428L/N434S; E233P/L234V/L235A/G236+A327G/A330S/P331S; E333A; S239D/A330L/I332E; P257I/Q311; K326W/E333S; S239D/I332E/G236A; N297Q; K322A; S228P; L235E+E318A/K320A/K322A; L234A/L235A; and L234A/L235A/P329G mutations, which mutations are summarized and annotated in “Engineered Fc Regions”, published by InvivoGen (2011) and available online at www. invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

In addition, antibodies have a hinge sequence that is typically situated between the Fab and Fc region (but a lower section of the hinge may include an amino-terminal portion of the Fc region). By way of background, an immunoglobulin hinge acts as a flexible spacer to allow the Fab portion to move freely in space. In contrast to the constant regions, hinges are structurally diverse, varying in both sequence and length between immunoglobulin classes and even among subclasses. For example, a human IgG1 hinge region is freely flexible, which allows the Fab fragments to rotate about their axes of symmetry and move within a sphere centered at the first of two inter-heavy chain disulfide bridges. By comparison, a human IgG2 hinge is relatively short and contains a rigid poly-proline double helix stabilized by four inter-heavy chain disulfide bridges, which restricts the flexibility. A human IgG3 hinge differs from the other subclasses by its unique extended hinge region (about four times as long as the IgG1 hinge), containing 62 amino acids (including 21 prolines and 11 cysteines), forming an inflexible poly-proline double helix and providing greater flexibility because the Fab fragments are relatively far away from the Fc fragment. A human IgG4 hinge is shorter than IgG1 but has the same length as IgG2, and its flexibility is intermediate between that of IgG1 and IgG2. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

In certain embodiments, the immunoglobulin binding protein comprises an antibody or an antigen-binding portion thereof. In particular embodiments, the antibody or antigen-binding portion thereof comprises monoclonal antibody 3E8. In further embodiments, the antibody or antigen-binding portion thereof comprises monoclonal antibody 5G2. In still other embodiments, the antibody or antigen-binding portion thereof comprises monoclonal antibody 4E2. In any of the embodiments disclosed herein, the immunoglobulin binding protein may be a chimeric, humanized, or human antibody or antigen-binding portion thereof. Among the provided immunoglobulin binding proteins are antibody fragments. An “antibody fragment” refers to a molecule other than an intact antibody that comprises a portion of an intact antibody that binds to the antigen to which the intact antibody binds. Examples of antibody fragments include, but are not limited to: Fv; Fab; Fab; Fab′-SH; F(ab′)₂; diabodies; linear antibodies; single-chain antibody molecules (e.g., scFv); tandem scFv; scFv-Fc; tandem scFv-Fc; scFv dimer; scFv-zipper; Diabody-Fc; Diabody-CH3; scDiabodies; scDiabody-Fc; scDiabody-CH3; nanobodies; TandAbs; minibodies; miniantibodies; triabodies; tetrabodies; scFab; Fab-scFv; Fab-scFv-Fc; scFv-CH-CL-scFv; and F(ab′)2-scFv2.

In particular embodiments, antibodies are single-chain antibody fragments comprising a variable heavy chain region, a variable light chain region or both, such as scFvs.

Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, such as, for example, proteolytic digestion of an intact antibody and production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are produced by enzyme digestion of a naturally-occurring intact antibody. In some aspects, the antibody fragments (e.g., binding domains) comprise scFvs. In some embodiments, an scFv comprises a V_{L} domain that is at least 80, 85, 90, 91, 92, 93, 94, 95, 96, 97, 98, 99, or 99.9% identical to the amino acid sequence shown in any one of SEQ ID NOS:3, 10, and 16, and a V_{H} domain that is at least 80, 85, 90, 91, 92, 93, 94, 95, 96, 97, 98, 99, or 99.9% identical to the amino acid sequence shown in any one of SEQ ID NOS:2, 8, and 14.

Any scFv of the present disclosure may be engineered so that the C-terminal end of the V_{L} domain is linked by a short peptide sequence to the N-terminal end of the V_{H} domain, or vice versa (i.e., (N)V_{L}(C)-linker-(N)V_{H}(C) or (N)V_{H}(C)-linker-(N)V_{L}(C).

In certain embodiments, the binding domain comprises a scFv and the scFv comprises the V_{L} and V_{H} of monoclonal antibody 3E8. In particular embodiments, the scFv comprises or consists of an amino acid sequence of SEQ ID NO: 5 or 6.

In other embodiments, the binding domain comprises a scFv and the scFv comprises the V_{L} and V_{H} of monoclonal antibody 5G2. In certain embodiments, the scFv comprises or consists of an amino acid sequence of SEQ ID NO: 11 or 12.

In still other embodiments, the binding domain comprises a scFv and the scFv comprises the V_{L} and V_{H} of monoclonal antibody 4E2. In particular embodiments, the scFv comprises or consists of an amino acid sequence of SEQ ID NO: 17 or 18. In any of the presently disclosed embodiments, a scFv linker can comprise a glycine-serine amino acid chain having from one to about ten repeats of GlyₓSer_{y}, wherein x and y are each independently an integer from 0 to 10, provided that x and y are not both 0 (e.g., (Gly₄Ser)₂ (SEQ ID NO: 20), (Gly₃Ser)₂ (SEQ ID NO:21), Gly₂Ser, or a combination thereof, such as ((Gly₃Ser)₂Gly₂Ser) (SEQ ID NO:49).

In certain aspects, an immunoglobulin binding protein comprises a multi-specific binding protein, wherein the multi-specific binding protein comprises a binding domain that specifically binds to the tag peptide and a binding domain that specifically binds to at least one target that is not the tag peptide. In particular embodiments, the multi-specific binding protein comprises a bispecific binding protein. Formats for bispecific binding proteins include antibody fragments as described herein and encompass, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-IgGs, IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, and DVI-IgG (four-in-one). Formats for bispecific antibody fragments are known in the art and described in, for example, Spiess et al., Mol. Immunol. 67(2):95 (2015) and in Brinkmann and Kontermann, mAbs 9(2):182-212 (2017), the antibody and antibody-fragment formats of which are herein incorporated by reference. In certain embodiments, the bispecific binding protein binds the tag peptide and the at least one target that is not the tag peptide is an immune cell marker. In specific embodiments, the immune cell marker is CD3 or CD16. In some embodiments, the bispecific binding protein binds the strep-tag peptide and the at least one target that is not the strep-tag peptide is selected from an antigen associated with a disease or disorder; e.g., a CD19, CD20, CD22, ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Ra2, IL-11Ra, MAGE-A1, MAGE-A3, MAGE-A4, SSX-2, PRAME, HA-1, PSA, ephrin A2, ephrin B2, an NKG2D, NY-ESO-1, TAG-72, mesothelin, NY-ESO, 5T4, BCMA, FAP, Carbonic anhydrase 9, ERBB2, BRAF^{V600E}, or CEA antigen.

In some embodiments, immunoglobulin binding proteins of the present disclosure are monovalent (i.e., have a single binding domain, which binding domain specifically binds the tag peptide) or multivalent (i.e., having more than one binding domain, at least one of which binding domains specifically binds the tag peptide), in which case they can be multispecific. In certain embodiments, the immunoglobulin binding protein is multivalent. In particular embodiments, the immunoglobulin binding protein is bivalent.

In some aspects, an immunoglobulin binding protein of the present disclosure is comprised in a fusion protein. In certain embodiments, the fusion protein comprises an extracellular component comprising a binding domain as disclosed herein, and an intracellular component comprising an effector domain, wherein the extracellular component and the intracellular component are connected by a transmembrane domain. In further embodiments, the binding domain comprises a scFv and the extracellular component further comprises a connector region comprising a hinge.

As used herein, an “effector domain” is an intracellular portion or domain of a fusion protein or receptor that can directly or indirectly promote a biological or physiological response in a cell when receiving an appropriate signal. In certain embodiments, an effector domain is from a protein or portion thereof or protein complex that receives a signal when bound, or when the protein or portion thereof or protein complex binds directly to a target molecule and triggers a signal from the effector domain.

An effector domain may directly promote a cellular response when it contains one or more signaling domains or motifs, such as an Intracellular Tyrosine-based Activation Motif (ITAM), as found in costimulatory molecules. Without wishing to be bound by theory, it is believed the ITAMs are important for T cell activation following ligand engagement by a T cell receptor or by a fusion protein comprising a T cell effector domain. In certain embodiments, the intracellular component comprises an ITAM. Exemplary effector domains include those from CD27, CD28, 4-1BB (CD137), OX40 (CD134), CD3ε, CD3 δ, CD3ζ, CD25, CD27, CD28, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In certain embodiments, an effector domain comprises a lymphocyte receptor signaling domain CD3ζ).

In further embodiments, the intracellular component of the fusion protein comprises a costimulatory domain or portion thereof selected from CD27, CD28, 4-1BB (CD137), OX40 (CD134), or a combination thereof. In certain embodiments, the intracellular component comprises a CD28 costimulatory domain or portion thereof (which may optionally include a LL→GG mutation at positions 186-187 of the native CD28 protein (see Nguyen et al., Blood 102:4320, (2003)), a 4-1BB costimulatory domain or portion thereof, or both.

In certain embodiments, an effector domain comprises CD3ζ or a functional portion thereof. In further embodiments, an effector domain comprises a portion or a domain from CD27. In further embodiments, an effector domain comprises a portion or a domain from CD28. In still further embodiments, an effector domain comprises a portion or a domain from 4-1BB. In further embodiments, an effector domain comprises a portion or a domain from OX40.

An extracellular component and an intracellular component of the present disclosure are connected by a transmembrane domain. A “transmembrane domain”, as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In certain embodiments, the transmembrane domain comprises or is derived from a known transmembrane protein (i.e., a CD4 transmembrane domain, a CD8 transmembrane domain, a CD27 transmembrane domain, a CD28 transmembrane domain, or any combination thereof).

In certain embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker of the present disclosure can position the binding domain away from the surface of a host cell expressing the fusion protein to enable proper contact between the host cell and a target cell, antigen binding, and activation (Patel et al., Gene Therapy 6: 412-419, 1999). Linker length may be varied to maximize antigen recognition based on the selected target molecule, selected binding epitope, or antigen binding domain seize and affinity (see, e.g., Guest et al., J. Immunother. 28:203-11, 2005; PCT Publication No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂ (SEQ ID NO: 20), (Gly₃Ser)₂ (SEQ ID NO:21), Gly₂Ser, or a combination thereof, such as ((Gly₃Ser)₂Gly₂Ser) (SEQ ID NO:49).

Linkers of the present disclosure also include immunoglobulin constant regions (i.e., CH1, CH2, CH3, or CL, of any isotype) and portions thereof. In certain embodiments, the linker comprises a CH3 domain, a CH2 domain, or both. In certain embodiments, the linker comprises a CH2 domain and a CH3 domain. In further embodiments, the CH2 domain and the CH3 domain are each a same isotype. In particular embodiments, the CH2 domain and the CH3 domain are an IgG4 or IgG1 isotype. In other embodiments, the CH2 domain and the CH3 domain are each a different isotype. In specific embodiments, the CH2 comprises a N297Q mutation. Without wishing to be bound by theory, it is believed that CH2 domains with N297Q mutation do not bind FcγR (see, e.g., Sazinsky et al., PNAS 105(51):20167 (2008)). In certain embodiments, the linker comprises a human immunoglobulin constant region or a portion thereof.

In any of the embodiments described herein, a linker may comprise a hinge region or a portion thereof. Hinge regions are flexible amino acid polymers of variable length and sequence (typically rich in proline and cysteine amino acids) and connect larger and less-flexible regions of immunoglobulin proteins. For example, hinge regions connect the Fc and Fab regions of antibodies and connect the constant and transmembrane regions of TCRs. In certain embodiments, the linker comprises an immunoglobulin constant region or a portion thereof and a hinge region or a portion thereof. In certain embodiments, the linker comprises a glycine-serine linker comprising or consisting of the amino acid sequence shown in SEQ ID NO: 20, 21, or 49.

In certain embodiments, one or more of the extracellular component, the binding domain, the linker, the transmembrane domain, the intracellular component, or the costimulatory domain comprises junction amino acids. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a fusion protein (e.g., amino acid residues resulting from the use of a restriction enzyme site or self-cleaving peptide sequences during the construction of a polynucleotide encoding a fusion protein). For example, a transmembrane domain of a fusion protein may have one or more junction amino acids at the amino-terminal end, carboxy-terminal end, or both.

In some embodiments, a fusion protein of the present disclosure may further comprise a protein tag (also called a peptide tag or tag peptide herein), provided that the protein tag is not a strep-tag peptide. Protein tags are unique peptide sequences that are affixed or genetically fused to, or are a part of, a protein of interest and can be recognized or bound by, for example, a heterologous or non-endogenous cognate binding molecule or a substrate (e.g., receptor, ligand, antibody, carbohydrate, or metal matrix). Protein tags are useful for detecting, identifying, isolating, tracking, purifying, enriching for, targeting, or biologically or chemically modifying tagged proteins of interest, particularly when a tagged protein is part of a heterogenous population of cells (e.g., a biological sample like peripheral blood). In the provided fusion proteins, the ability of the tag(s) to be specifically bound by the cognate binding molecules is distinct from, or in addition to, the ability of the binding domain(s) to specifically bind the target molecule(s) (i.e., a tag peptide comprising or consisting of the amino acid sequence shown in SEQ ID NO: 19). In certain embodiments, the protein tag is a Myc tag, His tag, Flag tag, Xpress tag, Avi tag, Calmodulin tag, Polyglutamate tag, HA tag, Nus tag, S tag, X tag, SBP tag, Softag, V5 tag, CBP, GST, MBP, GFP, Thioredoxin tag, or any combination thereof.

In specific embodiments, the fusion protein comprises a chimeric antigen receptor or a T cell receptor. “Chimeric antigen receptor” (CAR) refers to a fusion protein of the present disclosure engineered to contain two or more naturally-occurring amino acid sequences linked together in a way that does not occur naturally or does not occur naturally in a host cell, which fusion protein can function as a receptor when present on a surface of a cell. CARs of the present disclosure include an extracellular portion comprising an antigen binding domain (i.e., obtained or derived from an immunoglobulin or immunoglobulin-like molecule, such as an scFv derived from an antibody or TCR specific for a cancer antigen, or an antigen binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016); Stone et al., Cancer Immunol. Immunother., 63(11):1163 (2014)).

“T cell receptor” (TCR) refers to an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like immunoglobulins, the extracellular portion of TCR chains (e.g., α-chain, β-chain) contain two immunoglobulin domains, a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. Also, like immunoglobulins, the variable domains contain complementary determining regions (CDRs; also referred to as hypervariable regions or HVRs) separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). Generally, the CDR3 of a TCR variable domain is the CDR that primarily contacts a peptide antigen, while CDRs 1 and 2 primarily contact the MHC. In certain embodiments, a TCR is found on the surface of T cells (or T lymphocytes) and associates with the CD3 complex. The source of a TCR as used in the present disclosure may be from various animal species, such as a human, mouse, rat, rabbit or other mammal.

“Major histocompatibility complex molecules” (MHC molecules) refer to glycoproteins that deliver peptide antigens to a cell surface. MHC class I molecules are heterodimers consisting of a membrane spanning a chain (with three a domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. An MHC molecule may be from various animal species, including human, mouse, rat, cat, dog, goat, horse, or other mammals.

Methods of making fusion proteins, including CARs, are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; Walseng et al., Scientific Reports 7:10713, 2017; and Brentj ens et al., 2007, Clin. Cancer Res. 13:5426, the techniques of which are herein incorporated by reference. Methods for producing engineered TCRs are described in, for example, Bowerman et al., Mol. Immunol., 46(15):3000 (2009), the techniques of which are herein incorporated by reference.

In certain embodiments, the antigen-binding fragment of the TCR comprises a single chain TCR (scTCR), which comprises both the TCR Vα and Vβ domains TCR, but only a single TCR constant domain (Cα or Cβ). In certain embodiments, the antigen-binding fragment of the TCR, or chimeric antigen receptor is chimeric (e.g., comprises amino acid residues or motifs from more than one donor or species), humanized (e.g., comprises residues from a non-human organism that are altered or substituted so as to reduce the risk of immunogenicity in a human), or human.

Methods useful for isolating and purifying recombinantly produced soluble immunoglobulin binding proteins or fusion proteins, by way of example, may include obtaining supernatants from suitable host cell/vector systems that secrete the soluble protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble protein described herein include batch cell culture, which is monitored and controlled to maintain appropriate culture conditions. Purification of the soluble protein may be performed according to methods described herein and known in the art and that comport with laws and guidelines of domestic and foreign regulatory agencies.

In some embodiments, an immunoglobulin binding protein or fusion protein as disclosed herein further comprises one or more of a cytotoxic agent (e.g., a chemotherapeutic agent or bacterial toxin), a radioisotope, a radiometal, or a detectable agent. Exemplary detectable agents include enzymes (e.g., a chromogenic reporter enzyme, such as horseradish peroxidase (HRP) or an alkaline phosphatase (AP)), dyes, (e.g., cyanin dye, coumarin, rhodamine, xanthene, fluorescein or a sulfonated derivative thereof, and fluorescent proteins, including those described by Shaner et al., Nature Methods (2005)), fluorescent labels or moieties (e.g., PE, Pacific blue, Alexa fluor, APC, and FITC) DNA barcodes (e.g., ranging from five up to 75 nucleotides long), and peptide tags, provided that the peptide tag does not comprise or consist of the amino acid sequence shown in SEQ ID NO:19. As used herein, “peptide tag” or “protein tag” or “non-strep tag peptide tag” or “non-strep tag protein tag” refers to a unique peptide sequence that: is affixed to, fused to, or part of a protein of interest (e.g., an immunoglobulin binding protein of the present disclosure); is not a strep-tag peptide; and is specifically bound by a heterologous or non-endogenous cognate binding molecule, which binding properties can be used to detect, identify, isolate or purify, track, enrich for, or target a tagged peptide or protein or cells expressing a tagged peptide or protein, particularly when a tagged peptide or protein is part of a heterogeneous population of proteins or other material, or when cells expressing a tagged peptide or protein are part of a heterogeneous population of cells (e.g., biological sample). Exemplary non-strep tag peptide tags include Myc tag, His tag, Flag tag, Xpress tag, Avi tag, Calmodulin tag, Polyglutamate tag, HA tag, Nus tag, S tag, X tag, SBP tag, Softag, V5 tag, CBP, GST, MBP, GFP, Thioredoxin tag, or any combination thereof.

In another aspect, the present disclosure provides a composition comprising an immunoglobulin binding protein or a fusion protein as described herein and a pharmaceutically acceptable carrier, diluent, or excipient. Pharmaceutically acceptable carriers for diagnostic and therapeutic use are well known in the pharmaceutical art, and are described, for example, in Remington's Pharmaceutical Sciences, Mack Publishing Co. (A. R. Gennaro (Ed.), 18^{th} Edition, 1990) and in CRC Handbook of Food, Drug, and Cosmetic Excipients, CRC Press LLC (S. C. Smolinski, ed., 1992). Exemplary pharmaceutically acceptable carriers include any adjuvant, carrier, excipient, glidant, diluent, preservative, dye/colorant, surfactant, wetting agent, dispersing agent, suspending agent, stabilizer, isotonic agent, solvent, emulsifier, or any combination thereof. For example, sterile saline and phosphate buffered saline at physiological pH can be suitable pharmaceutically acceptable carriers. Preservatives, stabilizers, dyes or the like may also be provided in the pharmaceutical composition. In addition, antioxidants and suspending agents may also be used. Pharmaceutical compositions may also contain diluents such as water, buffers, antioxidants such as ascorbic acid, low molecular weight polypeptides (less than about 10 residues), proteins, amino acids, carbohydrates (e.g., glucose, sucrose, dextrins), chelating agents (e.g., EDTA), glutathione, and other stabilizers and excipients. Neutral buffered saline or saline mixed with nonspecific serum albumin are exemplary diluents.

Also provided herein are kits comprising (a) an expression vector or a polynucleotide encoding a tag peptide that comprises or consists of the amino acid sequence shown in SEQ ID NO: 19, and optional reagents for transducing the vector or polynucleotide into a host cell; and (b) an immunoglobulin binding protein, fusion protein, composition, isolated polynucleotide, or expression vector of the present disclosure, and optional reagents for transducing the polynucleotide or expression vector into a host cell, and/or a host cell comprising the polynucleotide or expression construct, wherein the host cell expresses the encoded immunoglobulin binding protein or fusion protein.

In another aspect, a matrix composition is provided that comprises (i) a matrix composition comprising an immunoglobulin binding protein or fusion protein as disclosed herein, and (ii) a binding polypeptide that specifically binds to an immune co-stimulatory molecule, wherein the binding increases an activity level of the immune co-stimulatory molecule. In certain embodiments, the matrix composition further comprises alginate, basement membrane matrix, or a biopolymer, or any combination thereof.

In still another aspect, a device is provided, wherein the device comprises: (i) an immunoglobulin binding protein or fusion protein as disclosed herein; and (ii) a binding polypeptide that specifically binds to an immune co-stimulatory molecule, wherein the binding increases an activity level of the immune co-stimulatory molecule. In particular embodiments of the device, one or both of (i) and (ii) are disposed on a solid surface, an agarose bead, a resin, a 3D fabric matrix, or a bead.

### Polynucleotides, Vectors, and Host Cells

In certain aspects, nucleic acid molecules are provided that encode any one or more of the immunoglobulin binding proteins or fusion proteins described herein. In certain embodiments, a polynucleotide of the present disclosure comprises one or both of (a) a polynucleotide having at least 70%, at least 75%, at least 80%, at least 85%, at least 90%, at least 95%, or 100% identity to the nucleotide sequence set forth in any one of SEQ ID NOS:1, 7, and 13 and (b) a polynucleotide having at least 70%, at least 75%, at least 80%, at least 85%, at least 90%, at least 95%, or 100% identity to the nucleotide sequence set forth in any one of SEQ ID NOS:4, 9, and 15.

A polynucleotide encoding a desired immunoglobulin binding protein or fusion protein can be obtained or produced using recombinant methods known in the art using standard techniques, such as screening libraries from cells expressing a desired sequence or a portion thereof, by deriving a sequence from a vector known to include the same, or by isolating a sequence or a portion thereof directly from cells or tissues containing the same. Alternatively, a sequence of interest can be produced synthetically.

In any of the embodiments described herein, a polynucleotide of the present disclosure may be codon-optimized for a host cell containing the polynucleotide (see, e.g, Scholten et al., Clin. Immunol. 119:135-145 (2006)). As used herein, a “codon-optimized” polynucleotide is a heterologous polynucleotide having codons modified with silent mutations corresponding to the abundances of host cell tRNA levels.

In further aspects, expression constructs are provided, wherein the expression constructs comprise a polynucleotide of the present disclosure operably linked to an expression control sequence (e.g., a promoter). In certain embodiments, the expression construct is comprised in a vector for introduction into a host cell of interest (e.g., a B cell, a CHO cell, a HEK-293 cell, a T cell, a NK cell, or a NK-T cell). An exemplary vector may comprise a polynucleotide capable of transporting another polynucleotide to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding fusion proteins as described herein) are co-administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector. Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

The viral vector can, in certain embodiments, be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing CAR transgenes are known in the art and have been previously described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

In any of the embodiments described herein, a polynucleotide can further comprise a polynucleotide that encodes a self-cleaving polypeptide, wherein the polynucleotide encoding the self-cleaving polypeptide is located between the polynucleotide encoding the immunoglobulin binding protein or fusion protein and the polynucleotide encoding the marker.

In certain embodiments, a self-cleaving polypeptide comprises a 2A peptide from porcine teschovirus-1 (P2A; SEQ ID NO:40 or 41), Thosea asigna virus (T2A; SEQ ID NO:42 or 43), equine rhinitis A virus (E2A; SEQ ID NO:44 or 45), or foot-and-mouth disease virus (F2A)). Further exemplary nucleic acid and amino acid sequences the 2A peptides are set forth in, for example, Kim et al. (PLOS One 6:e18556, 2011, which 2A nucleic acid and amino acid sequences are incorporated herein by reference in their entirety).

Other vectors developed for gene therapy uses can also be used with the compositions and methods of this disclosure. Such vectors include those derived from baculoviruses and α-viruses (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

Construction of an expression vector that is used for genetically engineering and producing a fusion protein of interest can be accomplished by using any suitable molecular biology engineering techniques known in the art. To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the protein or peptide of interest.

Markers are sometimes used to identify or monitor expression of a heterologous polynucleotide by a host cell transduced with the same, or to detect cells expressing a fusion protein of interest. In certain embodiments, a polynucleotide further comprises a polynucleotide that encodes a marker. In certain embodiments, the polynucleotide encoding the marker is located 3′ of the polynucleotide encoding the immunoglobulin binding protein or the fusion protein. In other embodiments, the polynucleotide encoding the marker is located 5′ of the polynucleotide encoding the immunoglobulin binding protein or the fusion protein. Exemplary markers include green fluorescent protein, an extracellular domain of human CD2, a truncated human EGFR (huEGFRt; see Wang et al., Blood 118:1255 (2011)), a truncated human CD19 (huCD19t), a truncated human CD34 (huCD34t); or a truncated human NGFR (huNGFRt). In certain embodiments, the encoded marker comprises EGFRt, CD19t, CD34t, or NGFRt.

Immunoglobulin binding proteins and fusion proteins of the present disclosure can, in certain aspects, be expressed on the surface of a host cell or be secreted by or isolated from a host cell. A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

Accordingly, in certain embodiments, host cells are provided that comprise a polynucleotide or expression construct of the present disclosure, wherein the polynucleotide or the expression construct encodes the immunoglobulin binding protein or the fusion protein and the host cell expresses the encoded immunoglobulin binding protein or the encoded fusion protein. The polynucleotides or cloning/expression constructs encoding immunoglobulin binding proteins are introduced into suitable cells using any method known in the art, including transformation, transfection and transduction. Host cells include the cells (e.g., T cells or other immune cells) of a subject undergoing ex vivo cell therapy including, for example, ex vivo gene therapy, as well as allogeneic or syngeneic cells used in cell therapies.

In certain embodiments, the host cell transduced to express an immunoglobulin binding protein or fusion protein of this disclosure is a hematopoietic progenitor cell or a human immune system cell. As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻ CD117⁺ phenotype or those found in the thymus (referred to as progenitor thymocytes).

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and NK-T cells). Exemplary immune system cells include a B cell, a CD4⁺ T cell, a CD8⁺ T cell, a CD4⁻ CD8⁻ double negative T cell, a γδ T cell, a regulatory T cell, a natural killer cell (e.g., a NK cell or a NK-T cell), and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune system cell that matures in the thymus and produces T cell receptors (TCRs). T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells), stem cell memory T cells, and effector memory T cells (T_{EM}, decreased expression of CD62L, CCR7, CD28, CD45RA, and increased expression of CD127 as compared to naïve T cells or T_{CM}).

Effector T cells (T_{E}) refers to antigen-experienced CD8⁺ cytotoxic T lymphocytes that have decreased expression of CD62L, CCR7, and CD28, and are positive for granzyme and perforin as compared to T_{CM}. Helper T cells (T_{H}) are CD4⁺ cells that influence the activity of other immune cells by releasing cytokines. CD4⁺ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺ CD25⁺ (Foxp3⁺) regulatory T cells and Treg17 cells, as well as Tr1, Th3, CD8⁺ CD28⁻, and Qa-1 restricted T cells.

“Cells of T cell lineage” refer to cells that show at least one phenotypic characteristic of a T cell, or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺ CD8⁺ double positive; single positive CD4⁺ or CD8⁺; TCRαβ or TCR γδ; or mature and functional or activated T cells.

Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to immunoglobulin binding proteins and fusion proteins of the present disclosure.

Eukaryotic host cells contemplated as an aspect of this disclosure when harboring a polynucleotide, vector, or protein according to this disclosure include, in addition to a human immune cells (e.g., a human patient's own immune cells), VERO cells, HeLa cells, Chinese hamster ovary (CHO) cell lines (including modified CHO cells capable of modifying the glycosylation pattern of expressed multivalent binding molecules, see US Patent Application Publication No. 2003/0115614), COS cells (such as COS-7), W138, BHK, HepG2, 3T3, RIN, MDCK, A549, PC12, K562, HEK293 cells, HepG2 cells, N cells, 3T3 cells, Spodoptera frupperda cells (e.g., Sf9 cells), Saccharomyces cerevisiae cells, and any other eukaryotic cell known in the art to be useful in expressing, and optionally isolating, a protein or peptide according to this disclosure. Also contemplated are prokaryotic cells, including Escherichia coli, Bacillus subtilis, Salmonella typhimurium, a Streptomycete, or any prokaryotic cell known in the art to be suitable for expressing, and optionally isolating, a protein or peptide according to this disclosure. In isolating protein or peptide from prokaryotic cells, in particular, it is contemplated that techniques known in the art for extracting protein from inclusion bodies may be used. Host cells that glycosylate the immunoglobulin binding proteins and fusion proteins of this disclosure are contemplated.

Transformed or transfected host cells may be cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media can also contain such components as growth factors or serum, as required. The growth medium will generally select for cells containing the exogenously added polynucleotide by, for example, drug selection or deficiency in an essential nutrient which is complemented by the selectable marker carried on the expression vector or co-transfected into the host cell.

In embodiments, an immunoglobulin binding protein or fusion protein of this disclosure is expressed on the surface of a host cell such that binding to a tag peptide elicits an activity or response from the host cell. Such expressed proteins may be functionally characterized according to any of a large number of art-accepted methodologies for assaying host cell (e.g., T cell) activity, including determination of T cell binding, activation or induction and also including determination of T cell responses that are antigen-specific. Examples include determination of T cell proliferation, T cell cytokine release, antigen-specific T cell stimulation, WIC restricted T cell stimulation, CTL activity (e.g., by detecting ⁵¹Cr or Europium release from pre-loaded target cells), changes in T cell phenotypic marker expression, and other measures of T cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

In other aspects, kits are provided comprising (a) a vector or an expression construct as described herein and optional reagents for transducing the vector or the expression construct into a host cell, and (b) (i) an immunoglobulin binding protein, fusion protein, isolated polynucleotide, or expression vector as disclosed herein, and optional reagents for transducing the polynucleotide or expression vector into a host cell, and (c) a host cell of this disclosure.

### Uses

In further aspects, methods are provided for using immunoglobulin binding proteins or fusion proteins of the present disclosure to identify a cell or population of cells that express a tag peptide having (i.e., comprising or consisting of) the amino acid sequence shown in SEQ ID NO: 19. Such methods may be useful, for example, to determine whether tagged cells used in adoptive cell therapies were successfully transferred to a subject in need thereof, or whether the tagged cells proliferated, or persisted, or localized to sites of interest in a subject receiving the adoptive cell therapy. In certain embodiments, a method comprises (i) contacting a sample from a subject comprising one or more tagged cells with an immunoglobulin binding protein or fusion protein of the present disclosure, and (ii) detecting specific binding of the immunoglobulin binding protein or the fusion protein to the one or more tagged cells, thereby identifying one or more cells that express the tag peptide.

In other aspects, methods are provided for enriching for or isolating a tagged cell or population of tagged cells from a subject, wherein the methods comprise (i) contacting a sample from the subject comprising one or more cells that express on the cell surface a tag peptide comprising or consisting of the amino acid sequence shown in SEQ ID NO: 19 with an immunoglobulin binding protein or fusion protein as disclosed herein, and (ii) selecting or sorting for tagged cell(s) specifically bound by the immunoglobulin binding protein or the fusion protein, thereby enriching for or isolating one or more cells that express the tag peptide. Such methods may possess utility in efficiently sorting and isolating tagged cells of interest from a subject or subject sample (e.g., from whole blood, from PBMCs, or from a tumor tissue or site) for analysis or manipulation to, for example, inform or improve adoptive cell therapies using the tagged cells.

In certain embodiments, the tag peptide is contained in a cell surface protein expressed by the cell(s) to be identified or enriched for or isolated. In particular embodiments, the cell surface protein comprises a CAR or a TCR (such as may be used to target a disease-associated antigen in an adoptive cell therapy comprising cells expressing the CAR or TCR), a marker (e.g., a detectable marker expressed on the cell surface, such as a transduction marker selected from EGFRt, CD19t, CD34t, or NGFRt), or a combination thereof. In certain embodiments, the cell surface protein comprises a marker. In further embodiments, the marker comprises an EGFRt, a CD19t, a CD34t, or a NGFRt. Representative tagged chimeric effector molecules, such as CARs containing one or more tag peptides, are described in PCT Publication No. WO 2015/095895, the tags and tagged effector molecules of which are herein incorporated by reference. Exemplary tag peptides include Strep®-Tag (WRHPQFGG, SEQ ID NO:48) and its variant Strep®-Tag II (WSHPQFEK, SEQ ID NO:19), which bind the bacterial protein Streptavidin, and its derivative Strep-Tactin, with high affinity. See, e.g., U.S. Pat. No. 7,981,632 (Strep tags from which are incorporated herein by reference).

An immunoglobulin binding protein or fusion protein of the present disclosure may comprise a detectable moiety to assist or enable identifying, tracking, enriching for, or isolating the bound tagged cells. For example, a detectable moiety can comprise one or more of an enzyme, a dye, a fluorescent label, or a peptide tag, provided that the peptide tag does not comprise a strep-tag peptide (e.g., does not comprise a peptide tag comprising or consisting of the amino acid sequence shown in SEQ ID NO:19).

In some embodiments, the detectable moiety comprises an enzyme and the enzyme comprises a chromogenic reporter enzyme, such as horseradish peroxidase or alkaline phosphatase. Fluorescent labels that may be coupled to an immunoglobulin binding protein or fusion protein of the present disclosure include cyanine dyes, coumarins, rhodamines, xanthenes, fluoresceins or sulfonated derivatives thereof, fluorescent proteins, or any combination thereof. Peptide labels useful in the presently disclosed methods include Myc tag, His tag, Flag tag, Xpress tag, Avi tag, Calmodulin tag, Polyglutamate tag, HA tag, Nus tag, S tag, X tag, SBP tag, Softag, V5 tag, CBP, GST, MBP, GFP, Thioredoxin tag, or any combination thereof. In particular embodiments, the detectable moiety comprises a peptide tag and the peptide tag comprises a His-tag or Myc-tag. In embodiments, a fluorescent moiety may be selected from PE, Pacific blue, Alexa fluor, APC or FITC.

Additional detectable moieties useful in any of the presently disclosed methods and compositions, as well as related labeling strategies and imaging techniques (e.g., PET, Mill, NIR), include those disclosed in Friese and Wu, Mol. Immunol. 67(200):142-152 (2015) and Moek et al., J. Nucl. Med. 58:83S-90S (2017), all of which are incorporated herein by reference. In certain embodiments, the detectable moiety comprises a radionuclide, a radiometal, a MM contrast agent, a microbubble, a carbon nanotube, a gold particle, fluorodeoxyglucose, a chromophore, a radio-opaque marker, or any combination thereof. In some embodiments, the detectable moiety comprises a radionuclide selected from ⁶⁸Ga, ⁶⁴Cu, ⁸⁶Y, ⁸⁹Zr, ¹²⁴I, ^{99m}Tc, ¹²³I, ¹¹¹In, ¹⁷⁷Lu, ¹³¹I, ⁷⁶Br, ⁷⁸Zr, ¹⁸F, and ¹²⁴T. In certain such embodiments, a detectably labeled immunoglobulin binding protein or fusion protein further comprises a radionuclide chelator selected from maleimide-labeled DOTA, N-hydroxysuccinimide-DOTA, and desferrioxamine (DFO).

Detectably bound tagged cells can be identified, selected, sorted, enriched for, or isolated using known techniques. For example, in certain embodiments, the cell or population of cells is identified, selected, or sorted using flow cytometry. In some embodiments, the tagged cell or population of tagged cells that are specifically bound by the immunoglobulin binding protein or the fusion protein is enriched or isolated from other components of the sample by magnetic column chromatography.

In some embodiments, the tagged cells to be identified are contained in a sample from a subject. In certain embodiments, the sample is blood or tissue. In any of the embodiments disclosed herein, the subject may be a human.

In other aspects, methods are provided for activating an immune cell modified to express on its cell surface a tag peptide comprising or consisting of the amino acid sequence shown in SEQ ID NO: 19, wherein the methods comprise contacting the modified immune cell with an immunoglobulin binding protein or fusion protein of the present disclosure, under conditions and for a time sufficient to induce activation of the modified immune cell. Briefly, immune cells such as T cells require activation by external stimuli in order to perform immune response functions (e.g., release cytokines and cytotoxins to kill infected or cancerous cells; provide signals to recruit other immune cells). Priming T cells expressing CARs or TCRs for adoptive therapy is typically performed by exposing the expressed CAR or TCR to its cognate antigen, or by exposing the T cells to microbead-coupled antibodies that bind a costimulatory protein present on the cell surface (e.g., CD3 or CD28).

Immunoglobulin binding proteins and fusion proteins of the present disclosure can be used to activate tagged immune cells, such as, for example, by binding to a tag contained in a CAR or TCR expressed by the immune cell (e.g., a tag contained in a portion of the CAR or TCR that does not specifically bind to the cognate antigen, such as an extracellular hinge portion), thereby mimicking an antigen-binding event. This advantageously allows the immune cell to be activated independent of antigen recognition by the expressed CAR or TCR. Alternatively, an immunoglobulin binding protein or fusion protein of this disclosure may be used to bind to and bring a tagged immune cell into proximity with other reagents that activate the tagged immune cell (e.g., using microbeads that include an antibody of the present disclosure and an antibody that agonistically binds to a co-stimulatory molecule such as CD3 or CD28). Such approaches may be useful where the tag peptide is not contained in a protein that signals for activation (e.g., if the tag peptide is fused to a marker protein such as EGFRt). In certain embodiments, the tag peptide is contained in a cell surface protein expressed by the modified immune cell. In further embodiments, the cell surface protein comprises a CAR, a TCR, a marker, or a combination thereof; optionally wherein the marker is selected from an EGFRt, a CD19t, a CD34t, or a NGFRt. In some embodiments, the immunoglobulin binding protein or fusion protein is attached to a solid surface, such as a planar surface, agarose, resin, 3D fabric matrix, or a bead (e.g., a microbead or a nanobead).

In certain embodiments, the tagged immune cell is activated in vitro or ex vivo, such as prior to or following initial administration of the tagged immune cell in an adoptive therapy regimen. In further embodiments, a method for activating tagged immune cells comprises an additional step of expanding the population of the activated tagged immune cells (e.g., in a sample from a subject) prior to enrichment or isolation thereof. In any of the embodiments disclosed herein, the cells that express the tag peptide may be or comprise human T cells, NK cells, or NK-T cells.

Also provided herein are in vivo methods for local activation of a modified immune cell, wherein the methods comprise administering to a subject (i) a matrix composition or device comprising (a) an immunoglobulin binding protein or fusion protein of the present disclosure, and (b) a binding polypeptide specific for a co-stimulatory molecule (e.g., CD3, CD27, CD28, OX40, or CD137) and (ii) a modified immune cell expressing on its cell surface a tag peptide comprising or consisting of the amino acid sequence shown in SEQ ID NO: 19, wherein the tag peptide is contained in a CAR, a TCR, a marker, or a combination thereof; optionally wherein the marker is selected from an EGFRt, a CD19t, a CD34t, or a NGFRt, wherein association of (a) of the matrix composition of subpart (i) with the cell surface tag peptide activates the modified immune cell. In some embodiments, the binding polypeptide specific for a co-stimulatory molecule is comprised in a multispecific immunoglobulin binding protein or fusion protein in the matrix composition or device. Such in vivo methods may be useful, for example, to activate tagged immune cells at or near a desired site for cell activity, such as at or near a tumor site or a site of infection. In certain embodiments, the matrix composition comprises alginate, a basement membrane matrix, or a biopolymer. In any of the embodiments disclosed herein, the cells that express the tag peptide may be or comprise human T cells, NK cells, or NK-T cells. Administration of the immunoglobulin binding protein or fusion protein, the binding polypeptide, and the modified immune cell may be performed in any order, but will typically be performed contemporaneously or simultaneously.

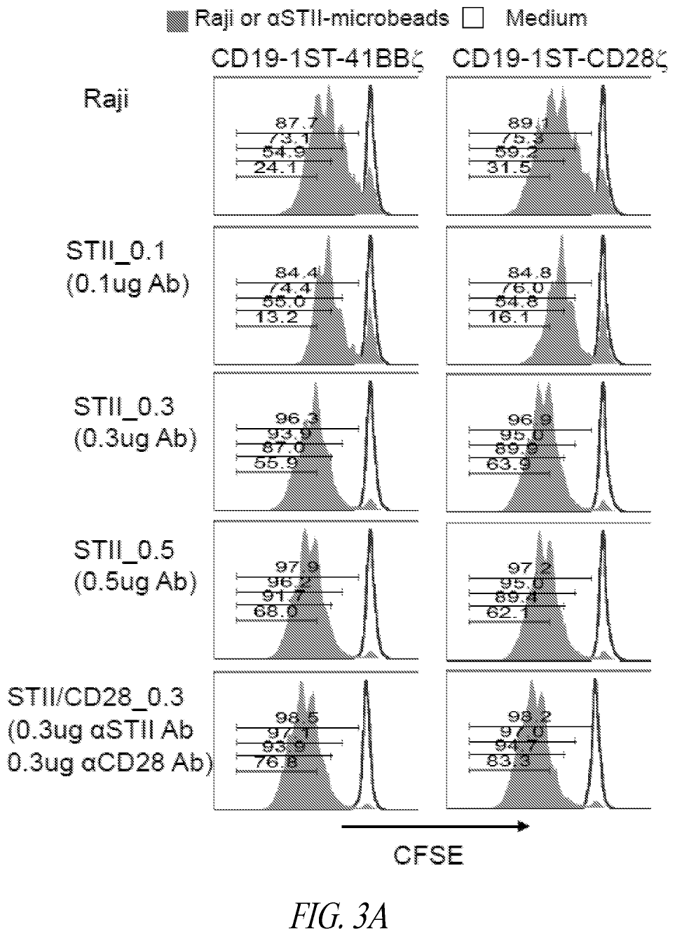

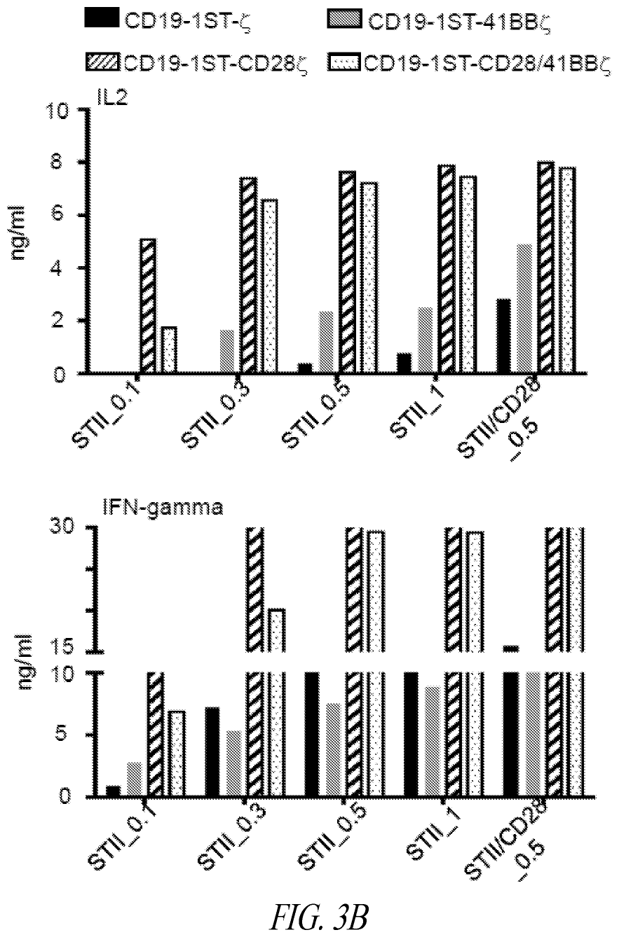

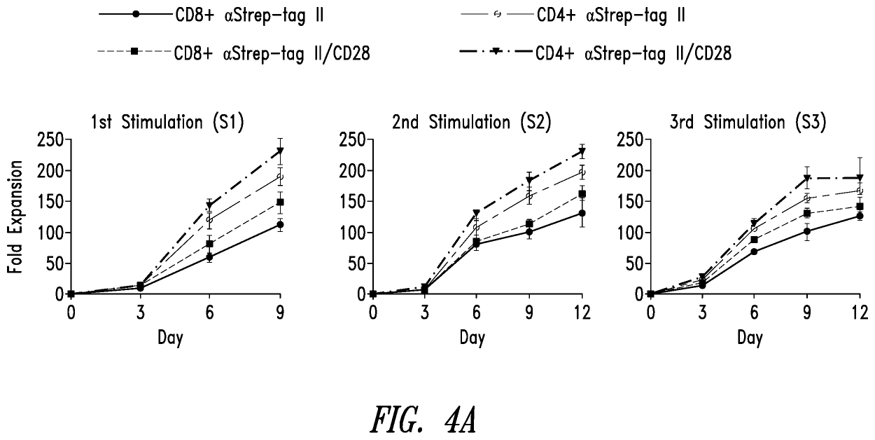

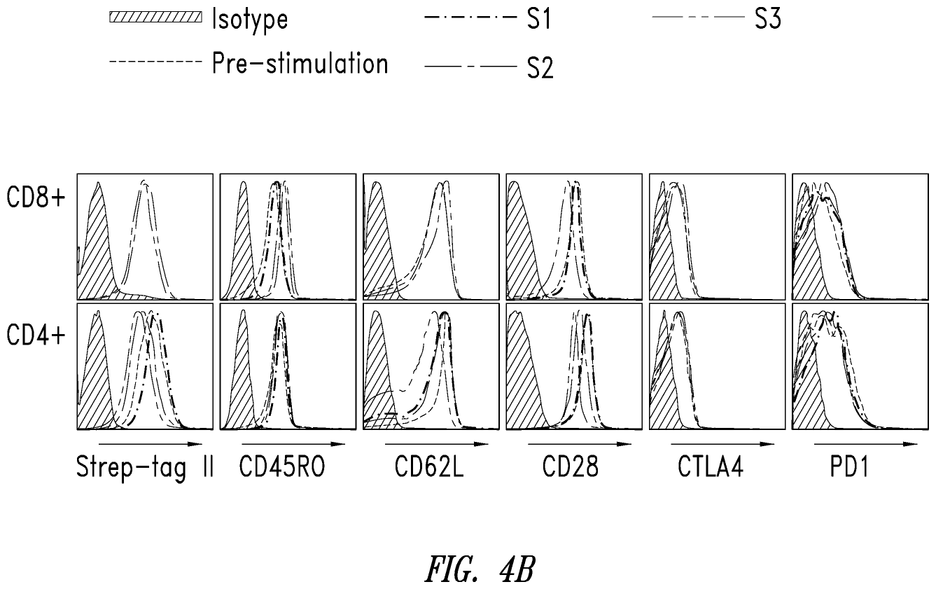

In yet another aspect, methods are provided for promoting cell proliferation, wherein the methods comprise contacting a cell expressing a tag peptide comprising or consisting of the amino acid sequence shown in SEQ ID NO: 19 with (a) an immunoglobulin binding protein or fusion protein of the present disclosure, and (b) a growth factor cytokine, under conditions and for a time sufficient to allow proliferation of the tagged cell. In certain embodiments, the immunoglobulin binding protein or fusion protein can promote proliferation by activating the cell, either directly (e.g., binding to a tag peptide contained in a CAR, a TCR, or a co-stimulatory molecule expressed by the cell) or indirectly (e.g., using a bead that comprises the immunoglobulin protein and an optional anti-CD3 or anti-CD28 antibody; see, e.g., FIGS. 3A-4B). In some embodiments, the immunoglobulin binding protein or fusion protein is attached to a solid surface, such as a planar surface, agarose, resin, 3D fabric matrix, or a bead (e.g., a microbead or a nanobead). Any growth factor cytokine may be used to promote cell proliferation according to the disclosed methods, provided that the growth factor cytokine stimulates cell proliferation. Such cytokines are known in the art and include, for example, IL-12, IL-15, and the like, and combinations thereof.

In further embodiments, the methods additionally comprise incubating the tagged cell with an agent that agonistically binds to a co-stimulatory protein expressed by the cell. In certain embodiments, the agent is an anti-CD27 binding protein, an anti-CD28 binding protein, an anti-CD137 binding protein, an anti-OX40 binding protein, or any combination thereof, wherein one or more of the binding proteins are attached to a solid surface. In further embodiments, the anti-CD27 binding protein, anti-CD28 binding protein, anti-CD137 binding protein, anti-OX40 binding protein, or any combination thereof, is attached to a planar surface, agarose, resin, 3D fabric matrix, or a bead.

The presently disclosed methods are useful for promoting proliferation of, for example, T cells, NK cells, or NK-T cells. In certain embodiments, the cell is a functional modified T cell. In specific embodiments, the functional modified T cell is a virus-specific cell, a tumor antigen specific cytotoxic T cell, a memory stem T cell, a central memory T cell, an effector T cell, or a CD4+CD25+ regulatory T cell. In still further embodiments, the tag peptide may contained in a cell surface protein expressed by the cell (e.g., a CAR, a TCR, a costimulatory molecule, a marker, or a combination thereof; optionally wherein the marker is selected from an EGFRt, a CD19t, a CD34t, or a NGFRt).

Proliferation of the tagged cells may be promoted or induced according to any of the presently disclosed methods in vitro, in vivo, or ex vivo. In particular embodiments, the proliferation is promoted or induced in vivo or ex vivo. Administration of the immunoglobulin binding protein or fusion protein and the growth factor cytokine may occur in any order (e.g., with the growth factor cytokine administered first), but will typically be performed simultaneously or contemporaneously.

In still other aspects, in vivo imaging methods are provided, wherein the methods comprise (a) administering, to a subject that has received modified cells expressing a tag peptide comprising or consisting of the amino acid sequence shown in SEQ ID NO: 19 and one or more of an immunoglobulin binding protein or a fusion protein of the present disclosure, wherein the immunoglobulin binding protein or fusion protein further comprises a detectable moiety suitable for in vivo imaging, and (b) performing imaging of the subject. Such methods are useful, for example, to track migration, localization, proliferation, or persistence of tagged cells in vivo.

Obtaining high-quality, informative images of cells in vivo depends on several factors, including, for example, the ability of the imaging agent to selectively bind the cells with high retention, to penetrate tissue(s) rapidly and to the necessary depth, and to be cleared rapidly from the blood. In embodiments, the detectably labeled immunoglobulin binding protein comprises a binding domain comprising an antigen-binding fragment of an antibody, wherein the antigen-binding fragment and has a format or structure that is amenable to in vivo imaging. For example, in certain embodiments, the antigen-binding fragment comprises a scFv, a tandem scFv, a scFv-Fc, a scFv dimer, a scFv zipper, a diabody, a minibody, a triabody, a tetrabody, a Fab, a F(ab)′2, a scFab, a miniantibody, a nanobody, a nanobody-HSA, a Bispecific T cell Engager (BiTE), a DART, a scDiabody, a scDiabody-CH3, or a scFv-CH3 Knobs-Into-Holes (KIH) assembly.

Any detectable moiety suitable for in vivo imaging can be used in the presently disclosed methods. In certain embodiments, the detectable moiety comprises a radioactive tracer, such as, for example, ⁶⁸Ga, ⁶⁴Cu, ⁸⁶Y, ⁸⁹Zr, ¹²⁴I, ^{99m}Tc, ¹²³I, ¹¹¹In, ¹⁷⁷Lu, ¹³¹I, ⁷⁶Br, ⁷⁸Zr, ¹⁸F, and ¹²⁴T. Positron Emission Tomography (PET) is an exemplary technique for imaging radioactively-labeled targets according to the instant methods. Additional imaging techniques useful for the in vivo imaging include, but are not limited to, magnetic resonance imaging (MM) and Near Infrared (NIR) imaging. Further detectable moieties include, for example, magnetic particles, superparamagnetic iron oxides (SPIO), fluorodeoxyglucose (18F), and fluorescent compounds such as fluorescent proteins or moieties.

In still other aspects, methods are provided for targeted ablation of tagged immunotherapy cells, wherein the methods comprise administering to a subject one or more of a presently disclosed (a) immunoglobulin binding protein, (b) fusion protein, or (c) composition, wherein the subject had previously been administered a tagged immunotherapy cell expressing cell surface protein (e.g., a CAR, TCR, or marker such as EGFRt) comprising a tag peptide, the tag peptide comprising or consisting of the amino acid sequence of SEQ ID NO: 19, wherein the immunoglobulin binding protein, fusion protein, or composition is capable of directly or indirectly inducing cell death upon binding to the tag peptide, under conditions and for a time sufficient to cause ablation of the tagged immunotherapy cells.

“Targeted ablation”, as used herein, refers to the selective killing (e.g., by induced apoptosis, lysis, phagocytosis, delivery of a cytotoxic agent, antibody-dependent cell-mediated toxicity (ADCC), complement-directed cytotoxicity (CDC), or by another mechanism) of target cells (e.g., cells expressing a tag peptide having the amino acid sequence shown in SEQ ID NO:19). Presently disclosed targeted ablation methods may be useful where the previously administered tagged immunotherapy cells (e.g., immunotherapy cells expressing an antigen-specific cell surface receptor such as a CAR or a TCR) are of an undesirably high number or have an undesirable activity (e.g., recognize and elicit an immune response against off-target cells or tissues in the subject) or level of activity (e.g., elicit an immune response of inappropriately high strength, duration, or both, such as a CRS event). In certain embodiments, an immunoglobulin binding protein, fusion protein, or composition is administered to the subject having at least one adverse event associated with the presence of the tagged immunotherapy cells.

In certain embodiments, the immunoglobulin binding protein, fusion protein, or composition comprises a cytotoxic agent, such as a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Reagents and chemistries for preparing a cytotoxic or detectable agent using a presently disclosed immunoglobulin binding protein (e.g., in an antibody-drug conjugate molecule) or fusion protein, and related mechanisms and methods, include those disclosed in Nareshkumar et al., Pharm. Res. 32:3526-3540 (2015), which compositions, methods, and techniques are incorporated herein by reference in their entirety. Click chemistries useful for generating protein-drug conjugates include those described in Meyer et al., Bioconjug. Chem. 27(12):2791-2807 (2016), and are incorporated herein by reference in their entirety.

Additional cytotoxic and detectable agents deliverable using a protein-drug conjugate include those disclosed in Parslow et al., Biomedicines 4:14 (2016), which agents and protein-drug conjugate design principles are incorporated herein by reference.

In further embodiments, the immunoglobulin binding protein comprises an antibody that is capable, upon binding to the tag peptide, of eliciting one or more of: (a) opsonization; (b) phagocytosis; (c) antibody-directed cell-mediated cytotoxicity (ADCC); and (d) complement-directed cytotoxicity (CDC) against the tagged immunotherapy cells.

Further, immunoglobulin binding proteins may be formatted to promote cell-mediated cytotoxicity against the tagged immunotherapy cells. For example, in certain embodiments, the immunoglobulin binding protein is bispecific and is capable of binding to (a) a T cell marker (e.g., CD3) or (b) an NK cell marker (e.g., CD28) at the same time as it binds to the tag peptide, thereby bringing a tagged cell into proximity with a T cell or NK cell to promote cytotoxic activity against the tagged cell. In specific embodiments, the immunoglobulin binding protein is bispecific and comprises a bispecific scFv, a bispecific T cell engager (BiTE) molecule, a Nanobody, a Diabody, DART, a TandAb, a scDiabody, a scDiabody-CH3, a Diabody-CH3, a Triple Body, a Miniantibody, a Minibody, a TriBi Minibody, a scFv-CH3 KIH, a Fab-scFv, a scFv-CH-CL-scFv, a F(ab′)2, a F(ab′)2-scFv2, a scFv-KIH, a Fab-scFv-Fc, a tetravalent HCab, a scDiabody-Fc, a Diabody-Fc, a tandem scFv-Fc, an Intrabody, a Dock and Lock fusion protein, a ImmTAC, a HSAbody, a scDiabody-HSA, a tandem scFv, a crossMab, a DAF (two-in-one or four-in-one), a DutaMab, a DT-IgG, a Knobs-Into-Holes (KIH) assembly, a KIH Common Light-Chain antibody, a Charge Pair, a Fab-arm Exchange, a SEEDbody, a Triomab, a LUZ-Y, a Fcab, a κλ-body, an orthogonal Fab, a DVD-IgG, an IgG(H)-scFv, a scFv-(H)IgG, an IgG(L)-scFv, a scFv-(L)IgG, an IgG(L,H)-Fv, an IgG(H)-V, a V(H)—IgG, an IgG(L)-V, a V(L)-IgG, a KIH IgG-scFab, a 2scFv-IgG, a IgG-2scFv, a scFv4-Ig, a Zybody, or a DVI-IgG (four-in-one).

In certain embodiments, the tagged immunotherapy cells were previously administered as a graft or a transplant (e.g., an organ or tissue graft or transplant), or for treating a disease such as a hyperproliferative disorder. As used herein, “hyperproliferative disorder” refers to excessive growth or proliferation as compared to a normal or undiseased cell. Exemplary hyperproliferative disorders include tumors, cancers, neoplastic tissue, carcinoma, sarcoma, malignant cells, pre malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like).

Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

In any of the foregoing embodiments, the cell surface protein comprises a chimeric antigen receptor (CAR), a T cell receptor (TCR), a marker, or a combination thereof. In certain embodiments, the cell surface protein comprises a marker. In particular embodiments, the marker comprises an EGFRt, a CD19t, a CD34t, or a NGFRt.

Ablation of the tagged immunotherapy cells may be determined necessary when the subject evidences one or more adverse effects associated with the tagged immunotherapy cells, such as graft-versus-host disease (GvHD), host-versus-graft disease (HvGD), or cytokine release syndrome (CRS) following a transplant or treatment comprising the tagged immunotherapy cells. Symptoms that may indicate a need for ablation of tagged immunotherapy cells include, for example, inflammation, fever, pulmonary or cerebral edema, changes in blood pressure or heart rate, undesirably low counts of healthy cells (e.g., white blood cells), undesirably high counts of tagged cells, elevated levels of cytokines, rash, blisters, jaundice, diarrhea, vomiting, abdominal cramps, fatigue, pain, stiffness, shortness of breath, weight loss, dry eyes or vision changes, dry mouth, vaginal dryness, and muscle weakness.

Targeted ablation of the tagged immunotherapy cells may be determined, either directly or indirectly, following treatment with the immunoglobulin binding protein, fusion protein, or composition. For example, in certain embodiments, a method further comprises, after the ablation: (i) performing in vivo imaging of the subject; (ii) performing a detection method in a sample obtained from the subject; (iii) monitoring a level of one or more cytokine (e.g., a pro-inflammatory cytokine, such as IL-12, IL-18, or IFN-γ) in the subject; (iv) detecting, in the subject or in a sample obtained from the subject, the presence and/or quantity of a target cell or tissue that was targeted by the tagged immunotherapy cells (e.g., B cells that were targeted by tagged anti-CD19 CAR T cells); (v) performing in vivo tracking of the tagged immunotherapy cells; or (vi) any combination thereof.

In vivo tracking of the tagged immunotherapy cells may be performed, for example, by using a conjugate comprising: (i) the immunoglobulin binding protein or fusion protein; and (ii) a magnetic particle, a superparamagnetic iron oxide (SPIO), fluorodeoxyglucose (18F), a fluorescent compound; or any combination thereof. In some embodiments, the in vivo tracking comprises use of Mill, PET, or near infrared imaging. Tagged immunotherapy cells that can be tracked in vivo using the present methods include T cells, NK cells, NK-T cells, hematopoietic stem cells, tissue cells, mesenchymal cells, or any combination thereof.

Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. An immunoglobulin binding protein, fusion protein, or composition according to the present disclosure may be administered in a manner appropriate to the disease, condition, or disorder to be treated as determined by persons skilled in the medical art. In any of the above embodiments, an immunoglobulin binding protein, fusion protein, or composition as described herein is administered intravenously, intraperitoneally, intratumorally, into the bone marrow, into a lymph node, or into the cerebrospinal fluid so as to encounter the tagged cells or tagged immunotherapy cells to be ablated. An appropriate dose, suitable duration, and frequency of administration of the compositions will be determined by such factors as a condition of the patient; size, type, and severity of the disease, condition, or disorder; the undesired type or level or activity of the tagged immunotherapy cells, the particular form of the active ingredient; and the method of administration.

Also contemplated are pharmaceutical compositions that comprise an immunoglobulin binding protein, fusion protein, or composition as disclosed herein and a pharmaceutically acceptable carrier, diluents, or excipient. Suitable excipients include water, saline, dextrose, glycerol, or the like and combinations thereof. In embodiments, the pharmaceutical compositions further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the tagged immunotherapy cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic or syngeneic) comprising a desired polynucleotide as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a desired, fusion protein-expressing T-cell composition to a subject as an adoptive immunotherapy. In certain embodiments, the host cell is a hematopoietic progenitor cell or a human immune cell. In certain embodiments, the immune system cell is a CD4⁺ T cell, a CD8⁺ T cell, a CD4⁻ CD8⁻ double negative T cell, a γδ T cell, a natural killer cell, a dendritic cell, or any combination thereof. In certain embodiments, the immune system cell is a naïve T cell, a central memory T cell, an effector memory T cell, or any combination thereof. In particular embodiments, the cell is a CD4+ T cell.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., an immunoglobulin binding protein, fusion protein, or composition with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of an immunoglobulin binding protein, fusion protein, or composition as described herein is administered to the subject, which may be administered at intervals between administrations of about two to about four weeks.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant and wherein the transplanted cells may optionally be tagged with a peptide comprising or consisting of the amino acid sequence shown in SEQ ID NO:19.

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The level of a CTL immune response may be determined by any one of numerous immunological methods described herein and routinely practiced in the art. The level of a CTL immune response may be determined prior to and following administration of any one of the herein described fusion proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, Pa.), pages 1127-50, and references cited therein).

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a tagged protein or cell as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

The pharmaceutical compositions described herein may be presented in unit-dose or multi-dose containers, such as sealed ampoules or vials. Such containers may be frozen to preserve the stability of the formulation until. In certain embodiments, a unit dose comprises a recombinant host cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine in the art and may be performed using samples obtained from a subject before and after treatment.

Methods according to this disclosure may further include administering one or more additional agents to treat the disease or disorder in a combination therapy. For example, in certain embodiments, a combination therapy comprises administering an immunoglobulin binding protein or fusion protein (or an engineered host cell expressing the same) or a composition, with (concurrently, simultaneously, or sequentially) an immune checkpoint inhibitor. In some embodiments, a combination therapy comprises administering an immunoglobulin binding protein, fusion protein, host cell, or composition of the present disclosure (or an engineered host cell expressing the same) with an agonist of a stimulatory immune checkpoint agent. In further embodiments, a combination therapy comprises administering an immunoglobulin binding protein, fusion protein, host cell, or composition of the present disclosure (or an engineered host cell expressing the same) with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GALS, KIR, PVR1G (CD112R), PVRL2, adenosine, A2aR, immunosuppressive cytokines (e.g., IL-10, IL-4, IL-1RA, IL-35), IDO, arginase, VISTA, TIGIT, LAIR1, CEACAM-1, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a fusion protein of the present disclosure (or an engineered host cell expressing the same) with one or more inhibitor of any one of the following immune suppression components, singly or in any combination.

In certain embodiments, an immunoglobulin binding protein, fusion protein, host cell, or composition used in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDI0680 (formerly AMP-514), AMP-224, BMS-936558 or any combination thereof. In further embodiments, an immunoglobulin binding protein, fusion protein, host cell, or composition is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof.

In certain embodiments, an immunoglobulin binding protein, fusion protein, host cell, or composition is used in combination with a LAG3 inhibitor, such as LAG525, IMP321, IMP701, 9H12, BMS-986016, or any combination thereof.

In certain embodiments, an immunoglobulin binding protein, fusion protein, host cell, or composition is used in combination with an inhibitor of CTLA4. In particular embodiments, an immunoglobulin binding protein, fusion protein, cell, or composition is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, an immunoglobulin binding protein, fusion protein, cell, or composition is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO 2016/40724 and WO 2013/025779.

In certain embodiments, an immunoglobulin binding protein, fusion protein, cell, or composition is used in combination with an inhibitor of CD244.

In certain embodiments, an immunoglobulin binding protein, fusion protein, cell, or composition is used in combination with an inhibitor of BLTA, HVEM, CD160, or any combination thereof. Anti CD-160 antibodies are described in, for example, PCT Publication No. WO 2010/084158.

In certain embodiments, an immunoglobulin binding protein, fusion protein, cell, or composition is used in combination with an inhibitor of TIM3.

In certain embodiments, an immunoglobulin binding protein, fusion protein, cell, or composition of the present disclosure is used in combination with an inhibitor of Gal9.

In certain embodiments, an immunoglobulin binding protein, fusion protein, cell, or composition is used in combination with an inhibitor of adenosine signaling, such as a decoy adenosine receptor.

In certain embodiments, an immunoglobulin binding protein, fusion protein, cell, or composition is used in combination with an inhibitor of A2aR.

In certain embodiments, an immunoglobulin binding protein, fusion protein, cell, or composition is used in combination with an inhibitor of KIR, such as lirilumab (BMS-986015).

In certain embodiments, an immunoglobulin binding protein, fusion protein, cell, or composition is used in combination with an inhibitor of an inhibitory cytokine (typically, a cytokine other than TGFβ) or Treg development or activity.

In certain embodiments, an immunoglobulin binding protein, fusion protein, cell, or composition is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, an immunoglobulin binding protein, fusion protein, cell, or composition is used in combination with an arginase inhibitor, such as N(omega)-Nitro-L-arginine methyl ester (L-NAME), N-omega-hydroxy-nor-1-arginine (nor-NOHA), L-NOHA, 2(S)-amino-6-boronohexanoic acid (ABH), S-(2-boronoethyl)-L-cysteine (BEC), or any combination thereof.

In certain embodiments, an immunoglobulin binding protein, fusion protein, cell, or composition is used in combination with an inhibitor of VISTA, such as CA-170 (Curis, Lexington, Mass.).

In certain embodiments an immunoglobulin binding protein, fusion protein, cell, or composition is used in combination with an inhibitor of TIGIT such as, for example, COM902 (Compugen, Toronto, Ontario Canada), an inhibitor of CD155, such as, for example, COM701 (Compugen), or both.

In certain embodiments, an immunoglobulin binding protein, fusion protein, cell, or composition is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, an immunoglobulin binding protein, fusion protein, cell, or composition is used in combination with a LAIR1 inhibitor.

In certain embodiments, an immunoglobulin binding protein, fusion protein, cell, or composition is used in combination with an inhibitor of CEACAM-1, CEACAM-3, CEACAM-5, or any combination thereof.

In certain embodiments, an immunoglobulin binding protein, fusion protein, cell, or composition is used in combination with an agent that increases the activity (i.e., is an agonist) of a stimulatory immune checkpoint molecule. For example, an immunoglobulin binding protein, fusion protein, cell, or composition can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof).

In any of the embodiments disclosed herein, a method may comprise administering an immunoglobulin binding protein, fusion protein, cell, or composition with one or more agonist of a stimulatory immune checkpoint molecule, including any of the foregoing, singly or in any combination.

In certain embodiments, a combination therapy comprises an immunoglobulin binding protein, fusion protein, cell, or composition and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering an immunoglobulin binding protein, fusion protein, cell, or composition and further comprises administering a radiation treatment or a surgery. Radiation therapy includes, for example, X-ray therapies, such as gamma-irradiation, and radiopharmaceutical therapies. Surgeries and surgical techniques appropriate to treating a given cancer or non-inflamed solid tumor in a subject are well-known to those of ordinary skill in the art.

In certain embodiments, a combination therapy method comprises administering an immunoglobulin binding protein, fusion protein, cell, or composition, and further comprises administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines are used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with the binding proteins or cells expressing the same of this disclosure.

### EXAMPLES

### Example 1

### Development and Characterization of Anti-STII Monoclonal Antibodies

Strep®-tag II (“STII”)-specific mAbs were developed using conventional immunization and hybridoma methods. Briefly, anti-STII monoclonal antibody hybridoma clones 4E2, 5G2, and 3E8 were generated by immunizing female 10-12 week old mice (BALB/c or CD1 or Swiss Webster or B57BL/6) with single STII peptides or with combinations of STII peptide sequences (Ac-C(dPEG4)NWSHPQFEK-amide (SEQ ID NO: 50), H2N-CGNWSHPQFEK-amide (SEQ ID NO: 51), H2N-CGNWSHPQFEKGC-OH (SEQ ID NO: 52)) maleimide coupled to KLH carrier protein. Following a 12-week boosting protocol with either Freund's Complete/Incomplete or Adjuplex adjuvants, splenocytes were isolated from high titer mice and electrofused to FoxNY myelomas (BTX, Harvard Apparatus). Hybridomas secreting peptide-specific antibody were identified and isolated using a ClonePix2 (Molecular Devices) colony picker. Antibodies from the picked clones were validated for peptide binding by flow cytometry using a cytometric bead array carrying the STII peptides, maleimide-coupled to BSA carrier protein. Selected hybridoma clones were subcloned using the ClonePix2 picker, with repeated validation for peptide binding. DNA sequences coding for IgGs were identified from multiple subclones derived from each clone (4E2, 5G2, and 3E8). Affinity purified IgG from the hybridomas was then further characterized in vitro and in vivo.

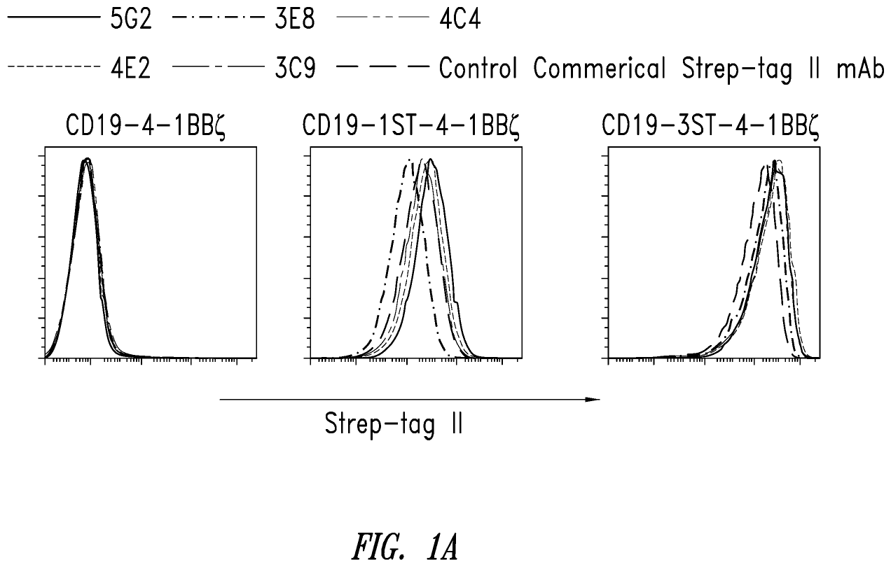

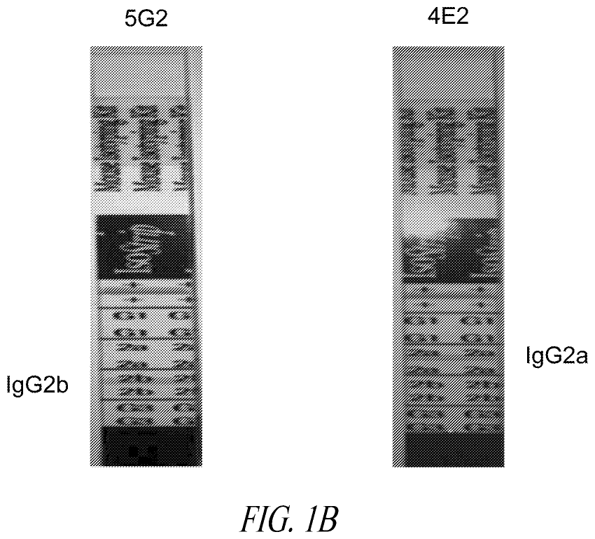

First, the antibodies were tested in vitro for specific binding to CAR T cells containing one (CD19-1ST-4-1BBζ), three (CD19-3ST-4-1BBζ), or zero (CD19-4-1BBz) STII tags. STII mAbs 4C4 and 3C9, obtained from a separate immunization, were also tested. CD19 Strep-tag CART cells (1ST-4-1BBζ and 3ST-41BBζ) and control cells (CD19-4-1BBζ) were stained with STII-specific mAb (5G2, 4E2, 3E8, 4C4, 3C9) or with a commercial STII-specific antibody (Genscript), followed by a FITC-conjugated goat anti-mouse secondary antibody. See FIG. 1A. These results show that STII mAbs of the present disclosure specifically target STII-tagged proteins such as STII-tagged CAR T cells.

Isotypes of 5G2 and 4E2 mAb were determined using a mouse isotyping kit (IsoStrip™, Sigma-Aldrich). The results show that 5G2 mAb is IgG2b and 4E2 mAb is IgG2a (FIG. 1B).

### Example 2

### In Vivo Targeting of Stii-Tagged CAR T Cells

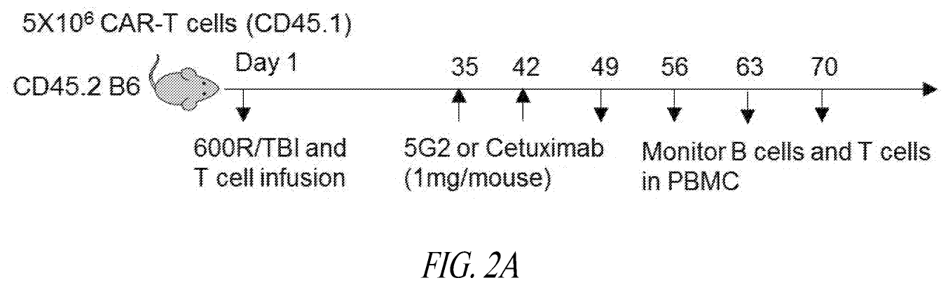

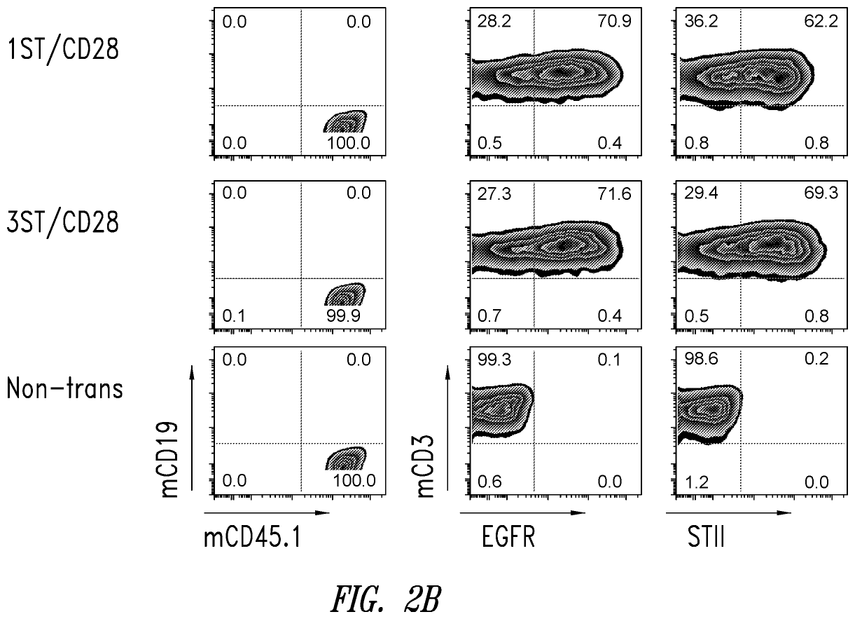

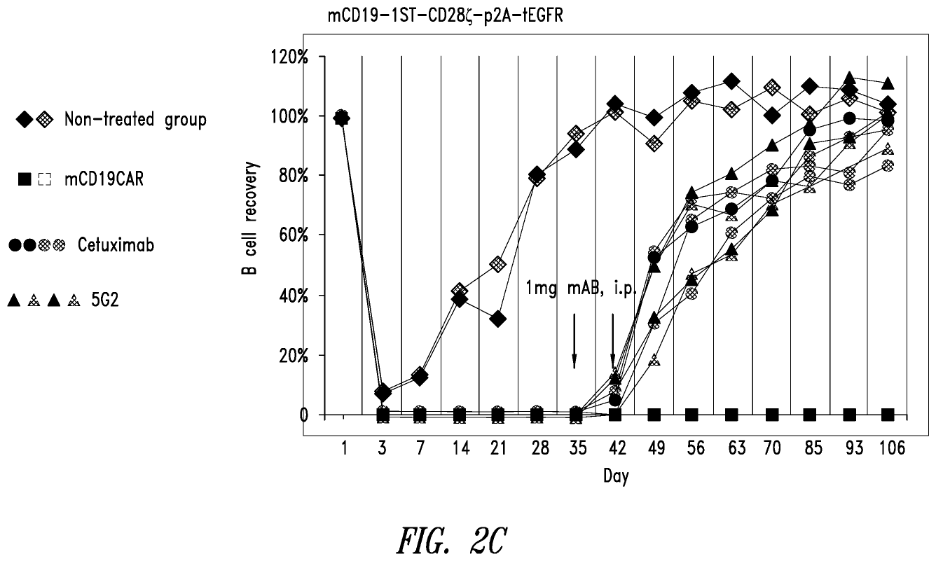

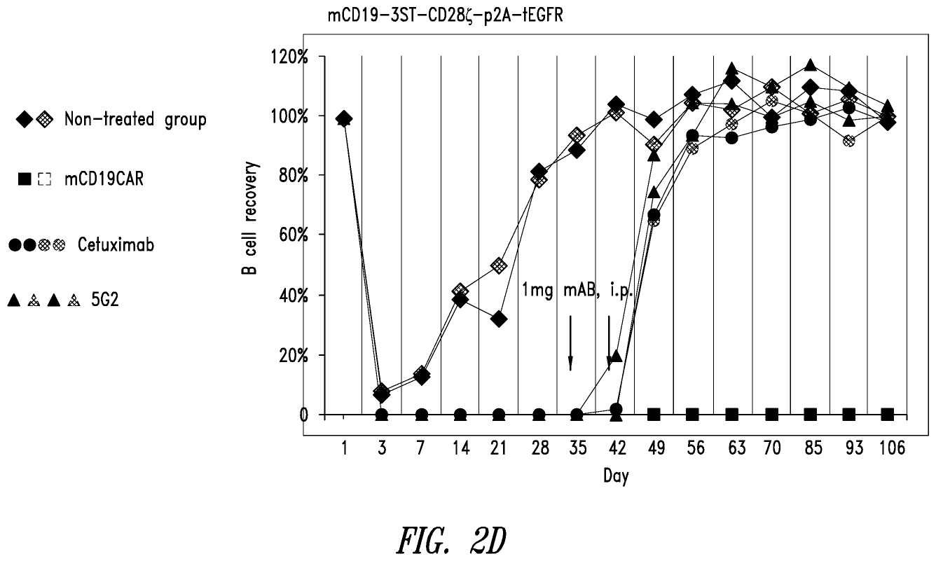

STII-specific antibodies may be useful for improving therapies that employ STII-tagged CAR T cells. To test whether an undesirable side effect associated with CAR T activity could be reduced or reversed by 5TH-specific antibodies, an in vivo B cell depletion assay was designed (FIG. 2A) wherein CD45.2⁺ C57/BL6 mice received sublethal radiation (6Gy TBI) and an infusion of 5×10⁶ CD45.1⁺ STII-tagged (1STII or 3STII) anti-CD19-CD2ζ_EGFRt CAR T cells in order to induce B cell aplasia. At Days +37 and +42, the mice were treated with anti-STII 5G2 mAb or Cetuximab (targeting the STII-CAR transduction marker EGFRt) 1 mg/mouse, i.p. As shown in FIG. 2B, EGFRt and STII-CAR expression in mouse T cells was measured at 7 days post-transduction using flow cytometry, prior to infusion of the transduced cells into recipient mice. These data show that both the 1STII-CAR and 3STII-CAR constructs were expressed by the T cells.

Mice then received treatment according to the schedule shown in FIG. 2A. T and B cell counts in PBMC from healthy and treated mice were monitored by flow cytometry over the course of the study. B cell recovery in mice that received radiation and anti-CD19-1STII CART cells, followed by antibody treatment, is shown in FIG. 2C. B cell recovery in mice that received radiation and anti-CD19-3STII CAR T cells, followed by antibody treatment, is shown in FIG. 2D.

Briefly, all groups of mice received 6Gy TBI on day 1. Non-treated group: mice were irradiated only and did not receive any T cell infusion; mCD19 CAR group: mice were infused with mCD19-1STII-CD28z or mCD193STII-CD28z CART cells on Day 1 and did not receive antibody treatment; Cetuximab group: mice received mCD19II-1ST-CD28z or anti-CD3STII-CD28z CART cells on Day 1 and Cetuximab (1 mg/mouse, i.p.) on Day 35 and Day 42; 5G2 group: mice received mCD19-1STII-CD28z or mCD19-3STII-CD28z CAR T cells on day 1 and anti-STII 5G2 mAb (1 mg/mouse, i.p.) on day 35 and day 42.

The results demonstrate that anti-STII mAbs are able to rescue B cell aplasia induced by mCD19-STII CAR-T cells as efficiently as Cetuximab, which targets the surrogate marker EGFRt on CAR-T cells.

### Example 3

### Functional Stimulation of STII-Tagged CAR T Cells

Anti-STII mAbs may also be useful for stimulating tagged CAR T cells; e.g., prior to infusion of tagged CAR T cells into a patient as an immunotherapy. CD19-1STII CART cells (CD28z or 4-1BBz) were stimulated using antigen-expressing Raji cells, antibody-coated microbeads (anti-STII at 0.1 μg, 0.3 m, or 0.5 m, or anti-STII and anti-CD28, both at 0.3 μg). The control was CD19-1STII CAR T cells in medium alone without antigen (Medium). Cells were labeled using carboxyfluorescein succinimidyl ester (CFSE). Proliferation was measured by FACS. As shown in FIG. 3A, anti-STII-coated microbeads stimulated proliferation, with the lowest level of anti-STII mAb having the greatest effect, as compared to medium alone. Cytokine production by the stimulated CAR T cells was measured. As shown in FIG. 3B, IL-2 and IFN-γ production by CAR T cells increased with the amount of anti-STII mAb present. The highest levels of cytokine production were by cells stimulated using anti-STII/anti-CD28-coated microbeads.

### Example 4

### Expansion of STII-Tagged Car T Cells

To further investigate the usefulness of anti-STII mAbs for priming tagged immunotherapy cells, CD8⁺ and CD4⁺ STII-containing CAR T cells were stimulated 3× using microbeads coated with anti-STII mAb alone or in combination with an anti-CD28 antibody.

As shown in FIG. 4A, each round of stimulation resulted in substantial expansion of the T cell population. Microbeads coated with both anti-STII and anti-CD28 had the greatest effect, and CD4⁺ CAR T cells expanded more than CD8⁺ cells.

### Example 5

### Characterization of Stimulated STII-Tagged CAR T Cells

CAR T cells (pre-stimulation, or after first, second, or third stimulation; see Example 4) were examined for expression of STII and several markers of T cell maturation, activation, or suppression (CD45RO, CD62L, CD28, CTLA4, and PD1). Cells were stained using antibodies for each marker and flow cytometry was performed. Data is shown in FIG. 4B.

Surprisingly, stimulated cells showed similar or even reduced expression of all markers as compared to pre-stimulation cells, even following the second and third stimulations. These data indicate that tagged CAR T cells can be efficiently expanded and stimulated in vitro using anti-STII mAbs of the present disclosure without an increased risk of T cell suppression or exhaustion.

The various embodiments described above can be combined to provide further embodiments. All of the U.S. patents, U.S. patent application publications, U.S. patent applications, foreign patents, foreign patent applications and non-patent publications referred to in this specification and/or listed in U.S. Provisional Patent Application No. 62/555,017 and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An immunoglobulin binding protein, wherein the binding protein comprises a binding domain that specifically binds to a tag peptide comprising or consisting of the amino acid sequence of SEQ ID NO:19 and the binding domain comprises:
(a) a V_{L} domain comprising:
i. a CDR1 amino acid sequence shown in SEQ ID NO:31, or a variant thereof, a CDR2 amino acid sequence shown in SEQ ID NO:32, or a variant thereof, and a CDR3 amino acid sequence shown in SEQ ID NO:33, or a variant thereof;
ii. a CDR1 amino acid sequence shown in SEQ ID NO:25, or a variant thereof, a CDR2 amino acid sequence shown in SEQ ID NO:26, or a variant thereof, and a CDR3 amino acid sequence shown in SEQ ID NO:27, or a variant thereof; or
iii. a CDR1 amino acid sequence shown in SEQ ID NO:37, or a variant thereof, a CDR2 amino acid sequence shown in SEQ ID NO:38, or a variant thereof, and a CDR3 amino acid sequence shown in SEQ ID NO:39, or a variant thereof, and a V_{H} domain; or
(b) a V_{H} domain comprising:
i. a CDR1 amino acid sequence shown in SEQ ID NO:28, or a variant thereof, a CDR2 amino acid sequence shown in SEQ ID NO:29, or a variant thereof, and a CDR3 amino acid sequence shown in SEQ ID NO:30, or a variant thereof;
ii. a CDR1 amino acid sequence shown in SEQ ID NO:22, or a variant thereof, a CDR2 amino acid sequence shown in SEQ ID NO:23, or a variant thereof, and a CDR3 amino acid sequence shown in SEQ ID NO:24, or a variant thereof; or
iii. a CDR1 amino acid sequence shown in SEQ ID NO:34, or a variant thereof, a CDR2 amino acid sequence shown in SEQ ID NO:35, or a variant thereof, and a CDR3 amino acid sequence shown in SEQ ID NO:36, or a variant thereof, and a V_{L} domain; or
(c) the V_{L} domain of (a) and the V_{H} domain of (b).

2. The immunoglobulin binding protein of claim 1, wherein the V_{L} domain comprises an amino acid sequence that is at least 80% identical to the amino acid sequence shown in any one of SEQ ID NOS: 3, 10, and 16, and the V_{H} domain comprises an amino acid sequence that is at least 80% identical to the amino acid sequence shown in any one of SEQ ID NOS: 2, 8, and 14.

3. (canceled)

4. The immunoglobulin binding protein of claim 2, wherein:
(i) the V_{L} comprises or consists of the amino acid sequence shown in SEQ ID NO:10, and the V_{H} comprises or consists of the amino acid sequence shown in SEQ ID No: 8;
(ii) the V_{L} comprises or consists of the amino acid sequence shown in SEQ ID NO:3, and the V_{H} comprises or consists of the amino acid sequence shown in SEQ ID NO:2; or
(iii) the V_{L} comprises or consists of the amino acid sequence shown in SEQ ID NO: 16, and the V_{H} comprises or consists of the amino acid sequence shown in SEQ ID NO:14.

5.-6. (canceled)

7. The immunoglobulin binding protein of claim 1, wherein the immunoglobulin binding protein comprises an antibody or an antigen-binding portion thereof.

8.-10. (canceled)

11. The immunoglobulin binding protein of claim 1, wherein the immunoglobulin binding protein is a chimeric, humanized, or human antibody or antigen-binding portion thereof.

12. The immunoglobulin binding protein of claim 1, wherein the binding domain comprises a scFv, a tandem scFv, a scFv-Fc, a tandem scFv-Fc, a scFv dimer, a scFv-zipper, a Diabody, a Diabody-Fc, a Diabody-CH3, a scDiabody, a scDiabody-Fc, a scDiabody-CH3, a Nanobody, a Minibody, a Miniantibody, a Triabody, a Tetrabody, a Fab, a F(ab)′2, a scFab, a Fab-scFv, a Fab-scFv-Fc, a scFv-CH-CL-scFv, a F(ab′)2-scFv2, a Bispecific T cell Engager (BiTE) molecule, a DART, a Knobs-Into-Holes (KIH) assembly, a scFv-CH3-KIH assembly, a KIH Common Light-Chain antibody, a TandAb, a Triple Body, a TriBi Minibody, a Fab-scFv, a scFv-CH-CL-scFv, a F(ab′)2-scFv2, a tetravalent HCab, an Intrabody, a CrossMab, a Dual Action Fab (DAF) (two-in-one or four-in-one), a DutaMab, a DT-IgG, a Charge Pair, a Fab-arm Exchange, a SEEDbody, a Triomab, a LUZ-Y, a Fcab, a κλ-body, an orthogonal Fab, a DVD-IgG, an IgG(H)-scFv, a scFv-(H)IgG, an IgG(L)-scFv, a scFv-(L)IgG, an IgG(L,H)-Fv, an IgG(H)-V, a V(H)—IgG, an IgG(L)-V, a V(L)-IgG, a KIH IgG-scFab, a 2scFv-IgG, a IgG-2scFv, a scFv4-Ig, a Zybody, or a DVI-IgG (four-in-one).

13.-18. (canceled)

19. The immunoglobulin binding protein of claim 1, wherein the immunoglobulin binding protein comprises a multi-specific binding protein, wherein the multi-specific binding protein comprises a binding domain that specifically binds to the tag peptide and a binding domain that specifically binds to at least one target that is not the tag peptide.

20. (canceled)

21. The immunoglobulin binding protein of claim 19, wherein the at least one target that is not the tag peptide is an immune cell marker.

22.-25. (canceled)

26. A fusion protein, comprising an extracellular component comprising the binding domain of claim 1, and an intracellular component comprising an effector domain, wherein the extracellular and intracellular components are connected by a transmembrane domain.

27.-28. (canceled)

29. The immunoglobulin binding protein of claim 1, further comprising a cytotoxic agent, radioisotope, radiometal, or detectable agent.

30. A composition, comprising (a) the immunoglobulin binding protein of claim 1, and a pharmaceutically acceptable carrier or excipient.

31. An isolated polynucleotide encoding (a) the immunoglobulin binding protein of claim 1.

32.-33. (canceled)

34. An expression construct, comprising the polynucleotide of claim 31 operably linked to an expression control sequence.

35. A vector, comprising the expression construct of claim 34.

36.-37. (canceled)

38. A host cell, comprising the polynucleotide of claim 31, wherein the host cell expresses the encoded immunoglobulin binding protein.

39. A method for identifying a tagged cell or a population of tagged cells that express on the cell surface a tag peptide comprising or consisting of the amino acid sequence shown in SEQ ID NO: 19, the method comprising:
(i) contacting a sample from a subject comprising one or more tagged cells with the immunoglobulin binding protein of claim 1; and
(ii) detecting specific binding of the immunoglobulin binding protein to the one or more tagged cells,
thereby identifying one or more cells that express the tag peptide.

40. A method for enriching for or isolating a tagged cell or population of tagged cells from a subject, the method comprising:
(i) contacting a sample from the subject comprising one or more cells that express on the cell surface a tag peptide comprising or consisting of the amino acid sequence shown in SEQ ID NO:19 with an immunoglobulin binding protein of claim 1; and
(ii) selecting or sorting the tagged cell(s) specifically bound by the immunoglobulin binding protein or the fusion protein,
thereby enriching for or isolating one or more cells that express the tag peptide.

41.-59. (canceled)

60. A method for activating an immune cell modified to express on its cell surface a tag peptide comprising or consisting of the amino acid sequence shown in SEQ ID NO: 19, the method comprising contacting the modified immune cell with an immunoglobulin binding protein of claim 1, under conditions and for a time sufficient to induce activation of the modified immune cell.

61.-71. (canceled)

72. A method for promoting cell proliferation, the method comprising contacting a cell expressing a tag peptide comprising or consisting of the amino acid sequence shown in SEQ ID NO:19, with:
(a) an immunoglobulin binding protein of claim 1, and
(b) a growth factor cytokine;
under conditions and for a time sufficient to allow proliferation of the tagged cell.

73.-85. (canceled)

86. An in vivo imaging method, the method comprising:
(a) administering, to a subject that has received modified cells expressing a tag peptide comprising or consisting of the amino acid sequence shown in SEQ ID NO: 19,
an immunoglobulin binding protein of claim 1,
wherein the immunoglobulin binding protein further comprises a detectable moiety suitable for in vivo imaging; and
(b) performing imaging of the subject.

87.-90. (canceled)

91. A method for targeted ablation of tagged immunotherapy cells, comprising administering to a subject
an immunoglobulin binding protein of claim 1,
wherein the subject had previously been administered a tagged immunotherapy cell expressing a cell surface protein comprising a tag peptide, the tag peptide comprising or consisting of the amino acid sequence of SEQ ID NO: 19,
wherein the immunoglobulin binding protein is capable of directly or indirectly inducing cell death upon binding the tag peptide,
under conditions and for a time sufficient to cause ablation of the tagged immunotherapy cells.

92.-110. (canceled)
